SEQUENCE LISTING (16 sequences)

SEQ ID NO: 1 - DNA, 624 bp, Homo sapiens
  CDS (1)..(624)
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccac

SEQ ID NO: 2 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 3 - DNA, 4913 bp, Homo sapiens
aaaaagagaaactgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggagcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgcaaaatgagttgtgatgaatcaatgaaaaatgtaatttagaaactgatttcttcagaattagatggcttattttttaaaatatttgaatgaaaacattttatttttaaaatattacacaggaggcttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacataaaaaaaaaaaaaaaaaaaaaaaaaaaaa

SEQ ID NO: 4 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 5 - DNA, 4261 bp, Homo sapiens
agccgccttcctatttccgcccggcgggcagcgctgcggggcgagtgccagcagagaggcgctcggtcctccctccgccctcccgcgccgggggcaggccctgcctagtctgcgtctttttcccccgcaccgcggcgccgctccgccactcgggcaccgcaggtagggcaggaggctggagagcctgctgcccgcccgcccgtaaaatggtcccctcggctggacagctcgccctgttcgctctgggtattgtgttggctgcgtgccaggccttggagaacagcacgtccccgctgagtgacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgagcatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttatttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaacggacactggtttttagacctctatcaatcagtagttagcatccaagagactttgcagaggcgtaggaatgaggctggacagatggcggaagcagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggatccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaatctttttttcctaaa

SEQ ID NO: 6 - protein, 159 aa, Homo sapiens
MVPSAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 7 - DNA, 1270 bp, Homo sapiens
agacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttggcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaagtattttttcaagttgtaaataatttatttaatatttaatggaagtgtatttattttacagctcattaaacttttttaaccaaacagaaaaaaaaaaaaaaaaaaaaaaaaaaaaaaaaa

SEQ ID NO: 8 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 9 - DNA, 1323 bp, Homo sapiens
gcccgaatatgtccctgggtgtgggtatgggtgtggggcaatttgggtgggagcagcgtggaggctcccaggaccaagtcctgcgcctctttggcggggtgtgtgcaggaggaggggggataaataggaggctccctcctcccggcgacattcacggagccggccggcctcccgccctgggtgtttccctgccttgtagccagggtgccagcctgggaagtagtttcgtttccttctgcctccgggattagtttccaggcaccctctcaggcgcccgaggcccgggaagggggcgaagaaggagggagacttgtctaggggctgcccggcccggcagagcggggttgatggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtttgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaaaaggctatgaagttacctccaggttggtggcaagctgcaaagtgccttgctcatttgaaaatggacagaatgtgtctcaggaaaacagctagtagacatgaattttaaataatgtatttactttttatttgcaactttagtttgtgttattattttttaataagaacattaattatatgtatattgtctagtaattgggaaaaaagcaactggttaggtagcaacaacagaagggaaatttcaataacctttcacttaagtattgtcaccaggattactagtcaaacaaaaaagaaaagtagaaaggaggttaggtcttaggaattgaattaataataaagctaccatttatcaagcatttaccatgtgctaataagtttgaaatatattatttcctttattcctttcagcaatccatgagatagctattataatcctc

SEQ ID NO: 10 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 11 - DNA, 4628 bp, Homo sapiens
tcacttgcctgatatttccagtgtcagagggacacagccaacgtggggtcccttctaggctgacagccgctctccagccactgccgcgagcccgtctgctcccgccctgcccgtgcactctccgcagccgccctccgccaagccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtgtgaagtgggttatactggtgtccgatgtgaacacttctttttaaccgtccaccaacctttaagcaaagaatatgtggctttgaccgtgattcttattattttgtttcttatcacagtcgtcggttccacatattatttctgcagatggtacagaaatcgaaaaagtaaagaaccaaagaaggaatatgagagagttacctcaggggatccagagttgccgcaagtctgaatggcgccatcaaacttatgggcagggataacagtgtgcctggttaatattaatattcccattttattaataatatttatgttgggtcaagtgttaggtcaataacactgtattttaatgtacttgaaaaatgtttttatttttgttttatttttgacagactatttgctaatgtataatgtgcagaaaatatttaatatcaaaagaaaattgatatttttatacaagtaatttcctgagctaaatgcttcattgaaagcttcaaagtttatatgcctggtgcacagtgcttagaagtaagcaattcccaggtcatagctcaagaattgttagcaaatgacagatttctgtaagcctatatatatagtcaaatcgatttagtaagtatgttttttatgttcctcaaatcagtgataattggtttgactgtaccatggtttgatatgtagttggcaccatggtatcatatattaaaacaataatgcaattagaatttgggagaagcaaatataggtcctgtgttaaacactacacatttgaaacaagctaaccctggggagtctatggtctcttcactcaggtctcagctataattctgttatatgaggggcagtggacagttccctatgccaactcacgactcctacaggtactagtcactcatctaccagattctgcctatgtaaaatgaattgaaaaacaattttctgtaatcttttatttaagtagtgggcatttcatagcttcacaatgttccttttttgtatattacaacatttatgtgaggtaattattgctcaacagacaattagaaaaaagtccacacttgaagcctaaatttgtgctttttaagaatatttttagactatttctttttataggggctttgctgaattctaacattaaatcacagcccaaaatttgatggactaattattattttaaaatatatgaagacaataattctacatgttgtcttaagatggaaatacagttatttcatcttttattcaaggaagttttaactttaatacagctcagtaaatggcttcttctagaatgtaaagttatgtatttaaagttgtatcttgacacaggaaatgggaaaaaacttaaaaattaatatggtgtatttttccaaatgaaaaatctcaattgaaagcttttaaaatgtagaaacttaaacacaccttcctgtggaggctgagatgaaaactagggctcattttcctgacatttgtttattttttggaagagacaaagatttcttctgcactctgagcccataggtctcagagagttaataggagtatttttgggctattgcataaggagccactgctgccaccacttttggattttatgggaggctccttcatcgaatgctaaacctttgagtagagtctccctggatcacataccaggtcagggaggatctgttcttcctctacgtttatcctggcatgtgctagggtaaacgaaggcataataagccatggctgacctctggagcaccaggtgccaggacttgtctccatgtgtatccatgcattatataccctggtgcaatcacacgactgtcatctaaagtcctggccctggcccttactattaggaaaataaacagacaaaaacaagtaaatatatatggtcatatacatattgtatatatattcatatacaaacatgtatgtatacatgaccttaatggatcatagaattgcagtcatttggtgctctgctaaccatttatataaaacttaaaaacaagagaaaagaaaaatcaattagatctaaacagttatttctgtttcctatttaatacagctgaagtcaaaatatgtaagaacacattttaaatactctacttacagttggccctctgtggttagttccacatctgtggattcaaccaaccaaggacggaaaatgcttaaaaaataatacaacaacaacaaaaaatacattataacaactatttacttttttttttttctttttgagatggagtctcgctctgttgcccaggttggagtgcagtggcacgatctcggctcactgcaacctcacctcccgggttcaagagatcctcctgcctcagcctcctgagcagctgggactacaggcgcatgccaccatgcccagctaatttttgtatttttagtagaggcggggtttcaccatgttggccaggatggtctcaatctcctaaccttgagatccaccctccacagcctcccaaactgctgggattacaggtgtgagccaccgcacgtagcatttacattaggtattacaagtaatgtaaagatgatttaagtatacaggaggatgtgaataggttatatgcaagcactatgcccttttatataagtgacttgaacatctgtgcccgattttagtatgtgcaggggggcgatctgggaatcagtcccctgtggataccaaggtacaactgtatttattaacgcttactagatgtgaggagagtctgaatattttcagtgatcttggctgtttcaaaaaaatctattgacttttcaataaatcagctgcaatccatttatttcatttacaaaagatttattgtaagcatctcaatcttggtttgtcagtttatcttaagcatgtcaattcataaaaacaagtcatttttgtatttttcatctttaagaatgcttaaaaaagctaatccctaaaatagttagatctttgtaaatgcatattaaataataaagtatgacccacattactttttatgggtgaaaataagacaaaaataatagttttagtgaggatggtgctgagtaaacataaaaactgatttgctctcagctgatgtgtcctgtacacagtgggaagattttagttcacacttagtctaactcccccattttacagatttctcactatatatatttctagaaggggctatgcatattcaatgtattgagaaccaaagcaaccacaaatgcataaatgcataatttatggtcttcaaccaaggccacataataacccagttaacttactctttaaccaggaatattaagttctataactagtactcaaggtttaaccttaaaattaagatttccttaaccttaaccttaaaattgatattatattaaacatacataatacaatgtaactccactgttctcctgaatattttttgctctaatctctctgccgaaagtcaaagtgatgggagaattggtatactggtatgactacgtcttaagtcagatttttatttatgagtctttgagactaaattcaatcaccaccaggtatcaaatcaacttttatgcagcaaatatatgattctagtgtctgacttttgttaaattcagtaatgcagtttttaaaaacctgtatctgacccactttgtaatttttgctccaatatccattctgtagacttttgaaaaaaaagtttttaatttgatgcccaatatattctgaccgttaaaaaattcttgttcatatgggagaagggggagtaatgacttgtacaaacagtatttctggtgtatattttaatgtttttaaaaagagtaatttcatttaaatatctgttattcaaatttgatgatgttaaatgtaatataatgtattttctttttattttgcactctgtaattgcactttttaagtttgaagagccattttggtaaacggtttttattaaagatgctatggaacataaagttgtattgcatgcaatttgaagtaacttatttgactatgaatgttatcggattactgaattgtatcaatttgtttgtgttcaatatcagctttgataattgtgtaccttaagatattgaaggagaaaatagataatttacaagatattattaatttttatttatttttcttgggaattgaaaaaaattgaaataaataaaaatgcattgaacatcttgcattcaaaatcttcactgac

SEQ ID NO: 12 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 13 - DNA, 847 bp, Homo sapiens
cgtcagtctagaaggataagagaaagaaagttaagcaactacaggaaatggctttgggagttccaatatcagtctatcttttattcaacgcaatgacagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgttttactggttatactggagaaaggtgtgagcacttgactttaacttcatatgctgtggattcttatgaaaaatacattgcaattgggattggtgttggattactattaagtggttttcttgttattttttactgctatataagaaagaggtatgaaaaagacaaaatatgaagtcacttcatatgcaatcgtttgacaaatagttattcaggccctataatgtgtcaggcactgacatgtaaaatttttttaattaaaaaagagctgtaatctggcaaaaagtttctatgtaatatttttcatgccttttctcataaacccagacgagtggtaaaaatttgccttcagttgtaataggagagttcaaacgtacagtctcccttcaacctatctctgtctgcccatatcaaaattataaatgaggaggacagcaggccccaagaaagtagggactaagtatgtcttgttcaaaattgtatattcagtgacttacactatgcctagcacacaacacacactgagtaaatatttgttgagtgaaataaaatcaagaaacaagtaaaaactga

SEQ ID NO: 14 - protein, 133 aa, Homo sapiens
MALGVPISVYLLFNAMTALTEEAAVTVTPPITAQQADNIEGPIALKFSHLCLEDHNSYCINGACAFHHELEKAICRCFTGYTGERCEHLTLTSYAVDSYEKYIAIGIGVGLLLSGFLVIFYCYIRKRYEKDKI

SEQ ID NO: 15 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 16 - protein, 1210 aa, Homo sapiens [sequence omitted]

(19) United States
(12) Patent Application Publication
(10) Pub. No.: US 2013/0130984 A1
(43) Pub. Date: May 23, 2013

(54) Administration Of Heparin Binding Epidermal Growth Factor For The Protection Of Enteric Neurons

(71) Applicant: Nationwide Children's Hospital, Inc., Columbus, OH (US)

(72) Inventor: Gail E. Besner, Dublin, OH (US)

(73) Assignee: NATIONWIDE CHILDREN'S HOSPITAL, INC., Columbus, OH (US)

(21) Appl. No.: 13/670,005

(22) Filed: Nov. 6, 2012

Related U.S. Application Data
(63) Continuation of application No. 12/872,961, filed on Aug. 31, 2010, now abandoned.

(51) Int. Cl. A61K 38/18

(52) U.S. Cl. CPC A61K 38/1808
USPC 514/9.6

(57) ABSTRACT

The invention provides for methods of protecting neurons within the enteric nervous system (ENS) comprising administering an EGF receptor agonist, such as heparin-binding EGF (HB-EGF). These methods include reducing damage of ENS neurons in patient s suffering from an intestinal injury. In addition, the invention provides for increasing intestinal motility in a patient suffering from an intestinal injury comprising administering HB-EGF. The invention also provides for methods of inducing neurite growth within the ENS in a patient suffering from intestinal injury comprising administering HB-EGF.

### FIELD OF INVENTION

The invention provides for methods of protecting neurons within the enteric nervous system (ENS) comprising administering an EGF receptor agonist, such as heparin-binding EGF (HB-EGF). These methods include reducing damage of ENS neurons in patient suffering from an intestinal injury. In addition, the invention provides for increasing intestinal motility in a patient suffering from an intestinal injury comprising administering HB-EGF. The invention also provides for methods of inducing neurite growth within the ENS of a patient suffering from an intestinal injury comprising administering HB-EGF.

### BACKGROUND

Heparin-binding epidermal growth factor (HB-EGF) was first identified in the conditioned medium of cultured human macrophages and later found to be a member of the epidermal growth factor (EGF) family of growth factors (Higashiyama et al., Science. 251:936-9, 1991). It is synthesized as a transmembrane, biologically active precursor protein (proHB-EGF) composed of 208 amino acids, which is enzymatically cleaved by matrix metalloproteinases (MMPs) to yield a 14-20 kDa soluble growth factor (sHB-EGF). Pro-HB-EGF can form complexes with other membrane proteins including CD9 and integrin α3β1; these binding interactions function to enhance the biological activity of pro-HB-EGF. ProHB-EGF is a juxtacrine factor that can regulate the function of adjacent cells through its engagement of cell surface receptor molecules.

Like other family members, HB-EGF binds to the EGF receptor (EGFR; ErbB-1), inducing its phosphorylation. Unlike most EGF family members, HB-EGF has the ability to bind strongly to heparan. Cell-surface heparan-sulfate proteoglycans (HSPG) can act as low affinity, high capacity receptors for HB-EGF. HB-EGF is produced by many different cell types including epithelial cells, and it is mitogenic and chemotactic for smooth muscle cells, keratinocytes, hepatocytes and fibroblasts. HB-EGF exerts its mitogenic effects by binding and activation of EGF receptor subtypes ErbB-1 and ErbB-4 (Junttila et al., Trends Cardiovasc Med; 10:304-310, 2001).

However, while the mitogenic function of HB-EGF is mediated through activation of ErbB-1, its migration-inducing function involves the activation of ErbB-4 and the more recently described N-arginine dibasic convertase (NRDc, Nardilysin). This is in distinction to other EGF family members, such as EGF itself, transforming growth factor (TGF)-α and amphiregulin (AR), which exert their signal-transducing effects via interaction with ErbB-1 only. In fact, the NRDc receptor is completely HB-EGF-specific. The differing affinities of EGF family members for the different EGFR subtypes and for HSPG may confer different functional capabilities to these molecules in vivo. The combined interactions of HB-EGF with HSPG and ErbB-1/ErbB-4/NRDc may confer a functional advantage to this growth factor. Importantly, endogenous HB-EGF is protective in various pathologic conditions and plays a pivotal role in mediating the earliest cellular responses to proliferative stimuli and cellular injury.

Administration of EGF to prevent tissue damage after an ischemic event in the brains of gerbils has been reported in U.S. Pat. No. 5,057,494 issued Oct. 15, 1991 to Sheffield. The patent projects that EGF “analogs” having greater than 50% homology to EGF may also be useful in preventing tissue damage and that treatment of damage in myocardial tissue, renal tissue, spleen tissue, intestinal tissue, and lung tissue with EGF or EGF analogs may be indicated. However, the patent includes no experimental data supporting such projections.

The small intestine receives the majority of its blood supply from the superior mesenteric artery (SMA), but also has a rich collateral network such that only extensive perturbations of blood flow lead to pathologic states. VIIIa et al. (Gastroenterology, 110(4 Suppl): A372, 1996) reports that in a rat model of intestinal ischemia in which thirty minutes of ischemia are caused by occlusion of the SMA, pre-treatment of the intestines with EGF attenuated the increase in intestinal permeability compared to that in untreated rats. The intestinal permeability increase is an early event in intestinal tissue changes during ischemia. Multiple animal models, like that described in VIIIa et al., supra have been used to study the effects of ischemic injury to the small bowel. Since the small intestine has such a rich vascular supply, researchers have used complete SMA occlusion to study ischemic injury of the bowel. Animals that experience total SMA occlusion for long periods of time suffer from extreme fluid loss and uniformly die from hypovolemia and sepsis, making models of this type useless for evaluating the recovery from intestinal ischemia. Nevertheless, the sequence of morphologic and physiologic changes in the intestines resulting from ischemic injury has remained an area of intense examination.

Miyazaki et al., Biochem Biophys Res Comm, 226: 542-546 (1996) discusses the increased expression in a rat gastric mucosal cell line of HB-EGF and AR resulting from oxidative stress. The authors speculate that the two growth factors may trigger the series of reparative events following acute injury (apparently ulceration) of the gastrointestinal tract.

EGF family members are of interest as intestinal protective agents due to their roles in gut maturation and function. Infants with necrotizing enterocolitis (NEC) have decreased levels of salivary EGF, as do very premature infants (Shin et al., J Pediatr. Surg. 35:173-176, 2000; Warner et al., J. Pediatr. 150:358-6, 2007). Studies have demonstrated the importance of EGF in preserving gut barrier function, increasing intestinal enzyme activity, and improving nutrient transport (Warner et al., Semin. Pediatr. Surg. 14:175-80, 2005). EGF receptor (EGFR) knockout mice develop epithelial cell abnormalities and hemorrhagic necrosis of the intestine similar to neonatal NEC, suggesting that lack of EGFR stimulation may play a role in the development of NEC (Miettinen et al., Nature 376:337-41, 1995). Dvorak et al. have shown that EGF supplementation reduces the incidence of experimental NEC in rats, in part by reducing apoptosis, barrier failure, and hepatic dysfunction (Am J Physiol. Gastrointest. Liver Physiol. 282:G156-G164, 2002). Vinter-Jensen et al., investigated the effect of subcutaneously administered EGF (150 μg/kg/12 hours) in rats, for 1, 2 and 4 weeks, and found that EGF induced growth of small intestinal mucosa and muscularis in a time-dependent manner (Regul. Pept. 61:135-142, 1996). Several case reports of clinical administration of EGF also exist. Sigalet et al. administered EGF (100 μg/kg/day) mixed with enteral feeds for 6 weeks to pediatric patients with short bowel syndrome (SBS), and reported improved nutrient absorption and increased tolerance to enteral feeds with no adverse effects (J Pediatr Surg 40:763-8, 2005). Sullivan et al., in a prospective, double-blind, randomized controlled study that included 8 neonates with NEC, compared the effects of a 6-day continuous intravenous infusion of EGF (100 ng/kg/hour) to placebo, and found a positive trophic effect of EGF on the intestinal mucosa (Ped. Surg. 42:462-469, 2007). Palomino et al. examined the efficacy of EGF in the treatment of duodenal ulcers in a multicenter, randomized, double blind human clinical trial in adults. Oral human recombinant EGF (50 mg/ml every 8 h for 6 weeks) was effective in the treatment of duodenal ulcers with no side effects noted (Scand. J. Gastroenterol. 35:1016-22, 2000).

Enteral administration of E. coli-derived HB-EGF has been shown to decrease the incidence and severity of intestinal injury in a neonatal rat model of NEC, with the greatest protective effects found at doses of 600 or 800 μg/kg/dose (Feng et al., Semin. Pediatr. Surg. 14:167-74, 2005). In addition, HB-EGF is known to protect the intestines from injury after intestinal ischemia/reperfusion injury (El-Assal et al., Semin. Pediatr. Surg. 13:2-10, 2004) or hemorrhagic shock and resuscitation (El-Assal et al., Surgery 142:234-42, 2007).

The prevention and treatment of intestinal damage in the clinical setting continues to be a challenge in medicine. There exists a need in the art for methods of preventing and/or treating intestinal damage including damage to the neurons within the ENS. Because of its neuroprotective effect within the intestine, HB-EGF may represent a promising therapeutic strategy for treating, reducing and preventing neuron damage after or during intestinal injury or intestinal diseases.

### SUMMARY OF INVENTION

The enteric nervous system (ENS), located in the wall of the intestine, is the largest and the most complex division of the peripheral nervous system. The ENS consists of interconnected networks (myenteric plexuses and submucosal plexuses) containing axons and enteric glial cells. Gastrointestinal motility is regulated by the ENS. Impaired intestinal motility is an important cause of significant morbidity after many forms of intestinal injury, including NEC. The data presented herein suggests that HB-EGF may have important effects on the ENS. HB-EGF administration may alleviate intestinal dysmotility, a significant source of post-injury morbidity in premature babies. This may have very significant clinical implications in the preservation or promotion of post-injury intestinal motility.

The invention provides for methods of administering HB-EGF to patients suffering from an intestinal injury in order to protect the neurons of the ENS or increase intestinal motility. In addition, the invention provides for the clinical use of HB-EGF in the prevention or treatment of intestinal injury such as NEC.

Intragastric administration of HB-EGF to rats is known to lead to delivery of the growth factor to the entire GI tract including the colon within 8 hours. HB-EGF is excreted in the bile and urine after intragastric or intravenous administration (Feng et al., Peptides. 27(6):1589-96, 2006). In addition, intragastric administration of HB-EGF to neonatal rats and minipigs has no systemic absorption of the growth factor (unpublished data). These findings collectively support the clinical feasibility and safety of enteral administration of HB-EGF in protection of the intestines from injury.

The invention provides for methods of increasing intestinal motility in a patient suffering from intestinal injury comprising administering an EGF receptor agonist in an amount effective to increase intestinal motility.

In another embodiment, the invention provides for methods of reducing damage to neurons within the ENS in a patient suffering from intestinal injury comprising administering an EGF receptor agonist in an amount effective to protect neurons within the ENS.

The invention also provides for methods of protecting neurons within the ENS in a patient suffering from intestinal injury comprising administering an EGF receptor agonist in an amount effective to protect neurons within the ENS.

In a further embodiment, the invention provides for methods of inducing neurite growth within the ENS in a patient suffering from intestinal injury comprising administering an EGF receptor agonist in an amount effective to induce neurite growth.

In any of the preceding methods, the intestinal injury may be caused by a conditions that affects intestinal motility such as necrotizing enterocolitis, hemorrhagic shock and resuscitation, ischemia/reperfusion injury, intestinal inflammatory conditions, such as Crohn's disease and ulcerative colitis, and intestinal infections. In addition, patients suffering from any of the following exemplary conditions will benefit from any of the preceding methods: Hirschprung's Disease, intestinal neuronal dysplasia, intestinal dysmotility disorders, intestinal pseudo-obstruction (Ogilvie's Syndrome), irritable bowel syndrome and chronic constipation.

NEC is an example of an intestinal injury that affects intestinal motility. The onset of symptoms of NEC refers to the occurrence or presence of one or more of the following symptoms: temperature instability, lethargy, apnea, bradycardia, poor feeding, increased pregavage residuals, emesis (may be bilious or test positive for occult blood), abdominal distention (mild to marked), occult blood in stool (no fissure), gastrointestinal bleeding (mild bleeding to marked hemorrhaging), significant intestinal distention with ileus, small-bowel separation, edema in bowel wall or peritoneal fluid, unchanging or persistent “rigid” bowel loops, pneumatosis intestinalis, portal venous gas, deterioration of vital signs, evidence of septic shock and pneumoperitoneum.

The invention provides for methods of administering an EGF receptor agonist to any patient suffering from an intestinal injury. In one embodiment, the invention contemplates administering an EGF receptor agonist to an infant or a premature infant. The term “premature infant” (also known as a “premature baby” or a “preemie”) refers to babies born having less than 36 weeks gestation. In another embodiment, the invention provides for methods of administering an EGF receptor agonist to an infant having a low birth weight or a very low birth weight. A low birth weight is a weight less than 2500 g (5.5 lbs.). A very low birth weight is a weight less than 1500 g (about 3.3 lbs.). The invention also provides for methods of administering HB-EGF to infants having intrauterine growth retardation, fetal alcohol syndrome, drug dependency, prenatal asphyxia, shock, sepsis, or congenital heart disease.

The methods of the invention may utilize any EGF receptor agonist. An EGF receptor agonist refers to a molecule or compound that activates the EGF receptor or induces the EGF receptor to dimerize, autophosphorylate and initiate cellular signaling. For example, any of the methods of the invention may be carried out with an EGF receptor agonist such as an EGF product or an HB-EGF product.

The methods of the invention are carried out with a dose of an EGF receptor agonist that is effective to increase intestinal motility or effective to reduce ENS neuron damage or effective to protect ENS neurons or effective to induce neurite growth. Exemplary effective doses are 100 μg/kg dose, 105 μg/kg dose, 110 μg/kg dose, 115 μg/kg dose, 120 μg/kg dose, 125 μg/kg dose, 130 μg/kg dose, 135 μg/kg dose, 140 μg/kg dose, 200 μg/kg dose, 250 μg/kg dose, 300 μg/kg dose, 400 μg/kg dose, 500 μg/kg dose, 550 μg/kg dose, 570 μg/kg dose, 600 μg/kg dose, 800 μg/kg dose and 1000 μg/kg dose. Exemplary dosage ranges of EGF receptor agonist that is effective to reduce the onset or severity of NEC are 100-140 μg/kg, 100-110 μg/kg dose, 110-120 μg/kg dose, 120-130 μg/kg dose, 120-140 μg/kg dose and 130-140 μg/kg dose. For example, the dose may be administered within about the first hour following birth or injury, within about 2 hours following birth or injury, within about 3 hours following birth or injury, within about 4 hours following birth or injury, within about 5 hours following birth or injury, within about 6 hours following birth or injury, within about 7 hours following birth or injury, within about 8 hours following birth or injury, within about 9 hours following birth or injury, within about 10 hours following birth or injury, within about 11 hours following birth or injury, within about 12 hours after birth or injury, within about 13 hours after birth or injury, within about 14 hours after birth or injury, within about 15 hours after birth or injury, within about 16 hours after birth or injury, within about 17 hours after birth or injury, within about 18 hours after birth or injury, within about 19 hours after birth or injury, within about 20 hours after birth or injury, within about 21 hours after birth or injury, within about 22 hours after birth or injury, within about 23 hours after birth or injury, within about 24 hours after birth or injury, within about 36 hours after birth or injury, within about 48 hours after birth or injury or within about 72 hours after birth or injury.

In one embodiment, an EGF receptor agonist is administered within about the first 12-72 hours after birth or injury. For example, the dose of an EGF receptor agonist may be administered about 12 hours after birth or injury, about 24 hours after birth or injury, about 36 hours after birth or injury, about 48 hours after birth or injury or about 72 hours after birth or injury. In further embodiments, the dose may be administered between hours 1-4 following birth or injury or between hours 2-5 following birth or injury or between hours 3-6 following birth or injury or between hours 4-7 following birth or injury or between hours 5-8 following birth or injury or between hours 6-9 following birth or injury or between hours 7-10 following birth or injury or between hours 8-11 following birth or injury, between hours 9-12 following birth or injury, between hours 10-13 following birth or injury, between hours 11-14 following birth or injury, between hours 12-15 following birth or injury, between hours 13-16 following birth or injury, between hours 14-17 following birth or injury, between hours 15-18 following birth or injury, between hours 16-19 following birth or injury, between hours 17-20 following birth or injury, between hours 18-21 following birth or injury, between hours 19-22 following birth or injury, or between hours 20-23 following birth or injury.

In another embodiment, an EGF receptor agonist is administered within 24 hours following the intestinal injury, such as administering an EGF receptor agonist within about the first 12-72 hours after injury. For example, the dose of an EGF receptor agonist may be administered about 12 hours following the injury, about 24 hours following the injury, about 36 hours following the injury, about 48 hours following the injury or about 72 hours following the injury. In further embodiments, the dose may be administered between hours 1-4 following the injury, between hours 21-24 following the injury, between hours 12-48 following the injury, between hours 24-36 following the injury, between hours 36-48 following the injury and between hours 48-72 following the injury or between hours 2-5 following the injury or between hours 3-6 following the injury or between hours 4-7 following the injury or between hours 5-8 following the injury or between hours 6-9 following the injury or between hours 7-10 following the injury or between hours 8-11 following the injury, between hours 9-12 following the injury, between hours 10-13 following the injury, between hours 11-14 following the injury, between hours 12-15 following the injury, between hours 13-16 following the injury, between hours 14-17 following the injury, between hours 15-18 following the injury, between hours 16-19 following the injury, between hours 17-20 following the injury, between hours 19-22 following the injury, or between hours 20-23 following the injury, between hours 21-24 following the injury, between hours 12-48 following the injury, between hours 24-36 following the injury, between hours 36-48 following the injury or between hours 48-72 following the injury.

The term “within 24 hours after birth” refers to administering at least a first unit dose of an EGF receptor agonist within about 24 hours following birth, and the first dose may be succeeded by subsequent dosing outside the initial 24 hour dosing period.

The term “within 24 hours after injury” refers to administering at least a first unit dose of an EGF receptor agonist within about 24 hours following the event causing the injury or damage to the intestine, and the first dose may be succeeded by subsequent dosing outside the initial 24 hour dosing period.

The EGF receptor agonist may be administered to the patient suffering the intestinal injury once a day (QD), twice a day (BID), three times a day (TID), four times a day (QID), five times a day (FID), six times a day (HID), seven times a day or 8 times a day. The EGF receptor agonist may be administered alone or in combination with feeding. The EGF receptor agonist may be administered to an infant with formula or breast milk with every feeding or a portion of feedings.

The methods of the invention may be carried out with any HB-EGF product including recombinant HB-EGF produced in E. coli and HB-EGF produced in yeast. The development of expression systems for the production of recombinant proteins is important for providing a source of protein for research and/or therapeutic use. Expression systems have been developed for both prokaryotic cells such as E. coli, and for eukaryotic cells such as yeast (Saccharomyces, Pichia and Kluyveromyces spp) and mammalian cells.

Intestinal motility in a patient suffering from intestinal injury may be assessed by monitoring and/or measuring abdominal distention, bloating, ability or failure to pass stool, vomiting, increased nasogastric tube output, cramping and abdominal pain and constipation.

Methods of measuring damage to ENS neurons in a patient suffering from intestinal injury include measuring intestinal motility studies, manometry studies, radiologic contrast studies including upper GI series and barium enema, and biopsy of the intestines.

### EGF Receptor Agonists

The Epidermal Growth Factor Receptor (EGFR) is a transmembrane glycoprotein that is a member of the protein kinase superfamily. The EGFR is a receptor for members of the epidermal growth factor family. Binding of the protein to a receptor agonist induces receptor dimerization and tyrosine autophosphorylation, and leads to cell proliferation and various other cellular effects (e.g. chemotaxis, cell migration).

The amino acid sequence of the EGF receptor is set out as SEQ ID NO: 16 (Genbank Accession No. NP_{—}005219). EGF receptors are encoded by the nucleotide sequence set out as SEQ ID NO: 15 (Genbank Accession No. NM_{—}005228). The EGF receptor is also known in the art as EGFR, ERBB, HER1, mENA, and PIG61. An EGF receptor agonist is a molecule that binds to and activates the EGF receptor so that the EGF receptor dimerizes with the appropriate partner and induces cellular signaling and ultimately results in an EGF receptor-induced biological effect, such as cell proliferation, cell migration or chemotaxis. Exemplary EGF receptor agonists include epidermal growth factor (EGF), heparin binding EGF (HB-EGF), transforming growth factor-α (TGF-α), amphiregulin, betacellulin, epiregulin, and epigen.

### Epidermal Growth Factor

Epidermal Growth Factor (EGF), also known as beta-urogastrone, URG and HOMG4, is a potent mitogenic and differentiation factor. The amino acid sequence of EGF is set out as SEQ ID NO: 4 (Genbank Accession No. NP_{—}001954). EGF is encoded by the nucleotide sequence set out as SEQ ID NO: 3 (Genbank Accession No. NM_{—}001963).

As used herein, “EGF product” includes EGF proteins comprising about amino acid 1 to about amino acid 1207 of SEQ ID NO: 4; EGF proteins comprising about amino acid 1 to about amino acid 53 of SEQ ID NO: 4; fusion proteins comprising the foregoing EGF proteins; and the foregoing EGF proteins including conservative amino acid substitutions. In a specific embodiment, the EGF product is human EGF (1-53), which is a soluble active polypeptide. Conservative amino acid substitutions are understood by those skilled in the art. The EGF products may be isolated from natural sources, chemically synthesized, or produced by recombinant techniques. In order to obtain EGF products of the invention, EGF precursor proteins may be proteolytically processed in situ. The EGF products may be post-translationally modified depending on the cell chosen as a source for the products.

The EGF products of the invention are contemplated to exhibit one or more biological activities of EGF, such as those described in the experimental data provided herein or any other EGF biological activity known in the art. For example, the EGF products of the invention may exhibit one or more of the following biological activities: cellular mitogenicity in a number of cell types including epithelial cells and smooth muscle cells, cellular survival, cellular migration, cellular differentiation, organ morphogenesis, epithelial cytoprotection, tissue tropism, cardiac function, wound healing, epithelial regeneration, promotion of hormone secretion such as prolactin and human gonadotrophin, pituitary hormones and steroids, and influence glucose metabolism.

The present invention provides for the EGF products encoded by the nucleic acid sequence of SEQ ID NO: 4 or fragments thereof including nucleic acid sequences that hybridize under stringent conditions to the complement of the nucleotides sequence of SEQ ID NO: 3, a polynucleotide which is an allelic variant of SEQ ID NO: 3; or a polynucleotide which encodes a species homolog of SEQ ID NO: 4.

### HB-EGF Polypeptide

The cloning of a cDNA encoding human HB-EGF (or HB-EHM) is described in Higashiyama et al., Science, 251: 936-939 (1991) and in a corresponding international patent application published under the Patent Cooperation Treaty as International Publication No. WO 92/06705 on Apr. 30, 1992. Both publications are hereby incorporated by reference herein in their entirety. In addition, uses of human HB-EGF are taught in U.S. Pat. No. 6,191,109 and International Publication No. WO 2008/134635 (Intl. Appl. No. PCT/US08/61772), also incorporated by reference in its entirety.

The sequence of the protein coding portion of the cDNA is set out in SEQ ID NO: 1 herein, while the deduced amino acid sequence is set out in SEQ ID NO: 2. Mature HB-EGF is a secreted protein that is processed from a transmembrane precursor molecule (pro-HB-EGF) via extracellular cleavage. The predicted amino acid sequence of the full length HB-EGF precursor represents a 208 amino acid protein. A span of hydrophobic residues following the translation-initiating methionine is consistent with a secretion signal sequence. Two threonine residues (Thr75 and Thr85 in the precursor protein) are sites for O-glycosylation. Mature HB-EGF consists of at least 86 amino acids (which span residues 63-148 of the precursor molecule), and several microheterogeneous forms of HB-EGF, differing by truncations of 10, 11, 14 and 19 amino acids at the N-terminus have been identified. HB-EGF contains a C-terminal EGF-like domain (amino acid residues 30 to 86 of the mature protein) in which the six cysteine residues characteristic of the EGF family members are conserved and which is probably involved in receptor binding. HB-EGF has an N-terminal extension (amino acid residues 1 to 29 of the mature protein) containing a highly hydrophilic stretch of amino acids to which much of its ability to bind heparin is attributed. Besner et al., Growth Factors, 7: 289-296 (1992), which is hereby incorporated by reference herein, identifies residues 20 to 25 and 36 to 41 of the mature HB-EGF protein as involved in binding cell surface heparin sulfate and indicates that such binding mediates interaction of HB-EGF with the EGF receptor.

As used herein, “HB-EGF product” includes HB-EGF proteins comprising about amino acid 63 to about amino acid 148 of SEQ ID NO: 2 (HB-EGF(63-148)); HB-EGF proteins comprising about amino acid 73 to about amino acid 148 of SEQ ID NO: 2 (HB-EGF(73-148)); HB-EGF proteins comprising about amino acid 74 to about amino acid 148 of SEQ ID NO: 2 (HB-EGF(74-148)); HB-EGF proteins comprising about amino acid 77 to about amino acid 148 of SEQ ID NO: 2 (HB-EGF(77-148)); HB-EGF proteins comprising about amino acid 82 to about amino acid 148 of SEQ ID NO: 2 (HB-EGF(82-148)); HB-EGF proteins comprising a continuous series of amino acids of SEQ ID NO: 2 which exhibit less than 50% homology to EGF and exhibit HB-EGF biological activity, such as those described herein; fusion proteins comprising the foregoing HB-EGF proteins; and the foregoing HB-EGF proteins including conservative amino acid substitutions. In a specific embodiment, the HB-EGF product is human HB-EGF (74-148). Conservative amino acid substitutions are understood by those skilled in the art. The HB-EGF products may be isolated from natural sources known in the art (e.g., the U-937 cell line (ATCC CRL 1593)), chemically synthesized, or produced by recombinant techniques such as disclosed in WO92/06705, supra, the disclosure of which is hereby incorporated by reference. In order to obtain HB-EGF products of the invention, HB-EGF precursor proteins may be proteolytically processed in situ. The HB-EGF products may be post-translationally modified depending on the cell chosen as a source for the products.

The HB-EGF products of the invention are contemplated to exhibit one or more biological activities of HB-EGF, such as those described in the experimental data provided herein or any other HB-EGF biological activity known in the art. One such biological activity is that HB-EGF products compete with HB-EGF for binding to the ErbB-1 receptor and has ErbB-1 agonist activity. In addition, the HB-EGF products of the invention may exhibit one or more of the following biological activities: cellular mitogenicity, cellular chemoattractant, endothelial cell migration, acts as a pro-survival factor (protects against apoptosis), decrease inducible nitric oxide synthase (iNOS) and nitric oxide (NO) production in epithelial cells, decrease nuclear factor-κB (NF-κB) activation, increase eNOS (endothelial nitric oxide synthase) and NO production in endothelial cells, stimulate angiogenesis and promote vasodilatation.

The present invention provides for the HB-EGF products encoded by the nucleic acid sequence of SEQ ID NO: 1 or fragments thereof including nucleic acid sequences that hybridize under stringent conditions to the complement of the nucleotides sequence of SEQ ID NO: 1, a polynucleotide which is an allelic variant of any SEQ ID NO: 1; or a polynucleotide which encodes a species homolog of SEQ ID NO: 2.

### Additional EGF Receptor Agonists

Additional EGF receptor agonists include: Transforming Growth Factor-α (TGF-α), also known as TFGA, which has the amino acid sequence set out as SEQ ID NO: 6 (Genbank Accession No. NP_{—}001093161), and is encoded by the nucleotide sequence set out as SEQ ID NO: 5 (Genbank Accession No. NM_{—}001099691); amphiregulin, also known as AR, SDGF, CRDGF, and MGC13647, which has the amino acid sequence set out as SEQ ID NO: 8 (Genbank Accession No. NP_{—}001648), and is encoded by the nucleotide sequence set out as SEQ ID NO: 7 (Genbank Accession No. NM_{—}001657); betacellulin (BTG) which has the amino acid sequence set out as SEQ ID NO: 10 (Genbank Accession No. NP_{—}001720), and is encoded by the nucleotide sequence set out as SEQ ID NO: 9 (Genbank Accession No. NM_{—}001729); Epiregulin (EREG), also known as ER, which has the amino acid sequence set out as SEQ ID NO: 12 (Genbank Accession No. NP_{—}001423) and is encoded by the nucleotide sequence set out as SEQ ID NO: 11 (Genbank Accession No. NM_{—}001432); and epigen (EPGN) also known as epithelial mitogen homolog, EPG, PRO9904, ALGV3072, FLJ75542, which has the amino acid sequence set out as SEQ ID NO: 14 (Genbank Accession No. NP_{—}001013460), and is encoded by the nucleotide sequence set out as SEQ ID NO: 13 (Genbank Accession No. NM_{—}001013442).

The EGF receptor agonists also may be encoded by nucleotide sequences that are substantially equivalent to any of the EGF receptor agonists polynucleotides recited above. Polynucleotides according to the invention can have at least, e.g., 65%, 70%, 75%, 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to the polynucleotides recited above. Preferred computer program methods to determine identity and similarity between two sequences include, but are not limited to, the GCG program package, including GAP (Devereux et al., Nucl. Acid. Res., 12: 387, 1984; Genetics Computer Group, University of Wisconsin, Madison, Wis.), BLASTP, BLASTN, and FASTA (Altschul et al., J. Mol. Biol., 215: 403-410, 1990). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

Included within the scope of the nucleic acid sequences of the invention are nucleic acid sequence fragments that hybridize under stringent conditions to any of SEQ ID NOS: 1, 3, 5, 7, 9, 11 and 13, or compliments thereof, which fragment is greater than about 5 nucleotides, preferably 7 nucleotides, more preferably greater than 9 nucleotides and most preferably greater than 17 nucleotides. Fragments of, e.g., 15, 17, or 20 nucleotides or more that are selective for (i.e., specifically hybridize to any one of the polynucleotides of the invention) are contemplated.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO4, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

The EGF receptor agonists of the invention include, but are not limited to, a polypeptide comprising: the amino acid sequences encoded by the nucleotide sequence of any one of SEQ ID NOS: 1, 3, 5, 7, 9, 11 and 13, or the corresponding full length or mature protein. In one embodiment, polypeptides of the invention also include polypeptides preferably with EGF receptor agonist biological activity described herein that are encoded by: (a) an open reading frame contained within any one of the nucleotide sequences set forth as SEQ ID NO: 1, 3, 5, 7, 9, 11 and 13, preferably the open reading frames therein or (b) polynucleotides that hybridize to the complement of the polynucleotides of (a) under stringent hybridization conditions. In another embodiment, polypeptides of the invention also include polypeptides preferably with EGF receptor agonist biological activity described herein that are encoded by: (a) an open reading frame contained within the nucleotide sequences set forth any as SEQ ID NO: 1, 3, 5, 7, 9, 11 and 13, preferably the open reading frames therein or (b) polynucleotides that hybridize to the complement of the polynucleotides of (a) under stringent hybridization conditions.

The EGF receptor agonists of the invention also include biologically active variants of any of the amino acid sequences of SEQ ID NO: 2, 4, 6, 8, 10, 12 and 14; and “substantial equivalents” thereof with at least, e.g., about 65%, about 70%, about 75%, about 80%, about 85%, 86%, 87%, 88%, 89%, at least about 90%, 91%, 92%, 93%, 94%, typically at least about 95%, 96%, 97%, more typically at least about 98%, or most typically at least about 99% amino acid identity) that retain EGF receptor agonist biological activity. Polypeptides encoded by allelic variants may have a similar, increased, or decreased activity compared to polypeptides having the amino acid sequence of any of SEQ ID NO: 2, 4, 6, 8, 10, 12 and 14.

The EGF receptor agonists of the invention include polypeptides with one or more conservative amino acid substitutions that do not affect the biological activity of the polypeptide. Alternatively, the EGF receptor agonist polypeptides of the invention are contemplated to have conservative amino acids substitutions which may or may not alter biological activity. The term “conservative amino acid substitution” refers to a substitution of a native amino acid residue with a normative residue, including naturally occurring and nonnaturally occurring amino acids, such that there is little or no effect on the polarity or charge of the amino acid residue at that position. For example, a conservative substitution results from the replacement of a non-polar residue in a polypeptide with any other non-polar residue. Further, any native residue in the polypeptide may also be substituted with alanine, according to the methods of “alanine scanning mutagenesis.” Naturally occurring amino acids are characterized based on their side chains as follows: basic: arginine, lysine, histidine; acidic: glutamic acid, aspartic acid; uncharged polar: glutamine, asparagine, serine, threonine, tyrosine; and non-polar: phenylalanine, tryptophan, cysteine, glycine, alanine, valine, proline, methionine, leucine, norleucine, isoleucine.

### Enteric Nervous System

The enteric nervous system (ENS), located in the wall of the intestine, is the largest and the most complex division of the peripheral nervous system. The ENS consists of interconnected networks (myenteric plexuses and submucosal plexuses) containing axons and enteric glial cells. Gastrointestinal motility is regulated by the ENS. The motility of the small intestine is considerably less organized in premature infants than in full term infants. It is thought that this intrinsic ENS immaturity makes preterm babies more vulnerable to NEC (Berseth et al., Pediatr. 115:646-51 (1989); Bernat et al., J. Lipid Mediat. 5:41-48 (1992)). In addition, post-NEC complications such as intestinal dysmotility, stricture, and recurrent abdominal distention have been wildly reported (Beardmore et al., Gastroenterology 74:914-7 (1978); Neu, J. Pediatr Clin North Am. 43:409-32 (1996), Dudgeon et al., J. Pediatr. Surg. 8:607-14 (1973), Boston et al., Pediatr. Surg. Int. 22:477-84 (2006).

The intestinal dysfunction that is present after either successful medical treatment of NEC, or aggressive surgical treatment of NEC, suggests that the compromised ENS is not fully recovered from the intestinal insult. The fact that delayed intestinal motility occurs after other insults to the GI tract illustrates that abnormal intestinal motility may be a sequallae of a variety of intestinal injury situations.

Impaired intestinal motility is an important cause of significant morbidity after many forms of intestinal injury, including NEC. Our preliminary data suggest that HB-EGF may have important effects on the ENS. HB-EGF administration may alleviate intestinal dysmotility, a significant source of post-injury morbidity in premature babies. This may have very significant clinical implications in the preservation or promotion of post-injury intestinal motility.

Other disorders that may cause intestinal injury and affect intestinal motility include hemorrhagic shock and resuscitation, ischemia/reperfusion injury, intestinal inflammatory conditions such as Crohn's disease and ulcerative colitis, intestinal infections, Hirschprung's Disease, intestinal dysmotility disorders, intestinal pseudo-obstruction (Ogilvie's Syndrome), irritable bowel syndrome and chronic constipation.

### Pharmaceutical Compositions

The administration of EGF receptor agonists is preferably accomplished with a pharmaceutical composition comprising an EGF receptor agonist and a pharmaceutically acceptable carrier. The carrier may be in a wide variety of forms depending on the route of administration. Suitable liquid carriers include saline, PBS, lactated Ringer solution, human plasma, human albumin solution, 5% dextrose and mixtures thereof. The route of administration may be oral, rectal, parenteral, or through a nasogastric or orogastric tube (enteral). Examples of parenteral routes of administration are intravenous, intra-arterial, intraperitoneal, intraluminally, intramuscular or subcutaneous injection or infusion.

The presently preferred route of administration of the present invention is the enteral route. Therefore, the present invention contemplates that the acid stability of HB-EGF is a unique factor as compared to, for example, EGF. For example, the pharmaceutical composition of the invention may also include other ingredients to aid solubility, or for buffering or preservation purposes. Pharmaceutical compositions containing EGF receptor agonists may comprise the agonist at a concentration of about 100 to 1000 μg/kg in saline. Suitable doses are in the range from 100-140 μg/kg, or 100-110 μg/kg, or 110-120 μg/kg, or 120-130 μg/kg, or 120-140 μg/kg, or 130-140 μg/kg, or 500-700 μg/kg, or 600-800 μg/kg or 800-1000 μg/kg. Preferred doses include 100 μg/kg, 120 μg/kg, 140 μg/kg and 600 μg/kg administered enterally once a day. Additional preferred doses may be administered once, twice, three, four, five, six or seven or eight times a day enterally.

The pharmaceutical compositions of EGF receptor agonist administered as methods of the invention include EGF receptor agonist which are associated or attached to carrier that assists in stabilizing the agonist during administration. For example, the invention contemplates administering HB-EGF associated with a carrier that prevent digestion in the duodenal fluids such as polymers, phospholipids, hydrogels, polysaccharides and prodrugs, microparticles or nanoparticles. The pharmaceutical compositions may also comprise a pH sensitive coatings or carriers for controlled release, pH independent biodegradable coatings or carriers or microbially controlled coatings or carriers.

The dose of EGF receptor agonist may also be administered intravenously. In addition, the dose of EGF receptor agonist may be administered as a bolus, either once at the onset of therapy or at various time points during the course of therapy, such as every four hours, or may be infused for instance at the rate of about 0.01 μg/kg/h to about 5 μg/kg/h during the course of therapy until the patient shows signs of clinical improvement. Addition of other bioactive compounds (e.g., antibiotics, free radical scavenging or conversion materials (e.g., vitamin E, beta-carotene, BHT, ascorbic acid, and superoxide dimutase), fibrolynic agents (e.g., plasminogen activators), and slow-release polymers) to the EGF receptor agonist or separate administration of the other bioactive compounds is also contemplated.

As used herein, “pathological conditions associated with intestinal ischemia” includes conditions which directly or indirectly cause intestinal ischemia (e.g., premature birth, birth asphyxia, congenital heart disease, cardiac disease, polycythemia, hypoxia, exchange transfusions, low-flow states, atherosclerosis, embolisms or arterial spasms, ischemia resulting from vessel occlusions in other segments of the bowel, ischemic colitis, and intestinal torsion such as occurs in infants and particularly in animals) and conditions which are directly or indirectly caused by intestinal ischemia (e.g., necrotizing enterocolitis, shock, sepsis, and intestinal angina). Thus, the present invention contemplates administration of an EGF receptor agonist to patients in need of such treatment including patients at risk for intestinal ischemia, patients suffering from intestinal ischemia, and patients recovering from intestinal ischemia. The administration of an EGF receptor agonist to patients is contemplated in both the pediatric and adult populations.

In view of the efficacy of HB-EGF in protecting neurons in the ENS, it is contemplated that HB-EGF has a similar protective effect on other segments of the peripheral nervous system and the central nervous system.

### Administration to Pediatric Patients

Intestinal injury related to an ischemic event is a major risk factor for neonatal development of necrotizing enterocolitis (NEC). NEC accounts for approximately 15% of all deaths occurring after one week of life in small premature infants. Although most babies who develop NEC are born prematurely, approximately 10% of babies with NEC are full-term infants. Babies with NEC often suffer severe consequences of the disease ranging from loss of a portion of the intestinal tract to the entire intestinal tract. At present, there are no known therapies to decrease the incidence of NEC in neonates.

Babies considered to be at risk for NEC are those who are premature (less than 36 weeks gestation) or those who are full-term but exhibit, e.g., prenatal asphyxia, shock, sepsis, or congenital heart disease. The presence and severity of NEC is graded using the staging system of Bell et al., J. Ped. Surg., 15:569 (1980) as follows:

| | |
|---|---|
| Stage I | Any one or more historical factors producing perinatal stress |
| (Suspected | Systemic manifestations—temperature instability, lethargy, |
| NEC) | apnea, bradycardia |
| | Gastrointestinal manifestations—poor feeding, increased |
| | pregavage residuals, emesis (may be bilious or test positive |
| | for occult blood), mild abdominal distention, occult blood in |
| | stool (no fissure) |
| Stage II | Any one or more historical factors |
| (Definite | Above signs and symptoms plus persistent occult or gross |
| NEC) | gastrointestinal bleeding, marked abdominal distention |
| | Abdominal radiographs showing significant intestinal |
| | distention with ileus, small-bowel separation (edema in |
| | bowel wall or peritoneal fluid), unchanging or persistent |
| | “rigid” bowel loops, pneumatosis intestinalis, portal venous |
| | gas |
| Stage III | Any one or more historical factors |
| (Advanced | Above signs and symptoms plus deterioration of vital signs, |
| NEC) | evidence of septic shock, or marked gastrointestinal |
| | hemorrhage |
| | Abdominal radiographs showing pneumoperitoneum in |
| | addition to findings listed for Stage II |

Babies at risk for or exhibiting NEC are treated as follows. Patients receive a daily liquid suspension of HB-EGF (e.g. about 1 mg/kg in saline or less). The medications are delivered via a nasogastric or orogastric tube if one is in place, or orally if there is no nasogastric or orogastric tube in place.

### BRIEF DESCRIPTION OF DRAWINGS

FIG. 1 depicts HB-EGF-induced neurite outgrowth in PC12 cells. Panel (A) depicts changes in cell shape and neurite outgrowth induced by HB-EGF and NGF. Scale bar=100 μm. Panel (B) provides quantification of neurite extension induced by HB-EGF. The percentage of cells with at least one neurite longer than the cell body diameter was determined 3 days after stimulation. Panel (C) depicts dose dependent response of PC12 cells to HB-EGF-mediated neurite outgrowth. Cells containing at least one neurite that was longer than the cell body diameter were counted, and the percentage of cells with neurites was determined. Values shown are mean±SEM of ˜100 cells obtained from three independent experiments. * p<0.01 vs. control; ** p<0.01 vs. HB-EGF; Φp<0.01 vs. NGF.

FIG. 2 depicts HB-EGF-induced activation of ERK, Ras and Rap1 in PC12 cells under non-injury conditions as detected by Western blot. The intensity of immunoreactive bands on Western blots was quantified. The band intensity ratio of phosphorylated Erk1/2 to total Erk1/2, Ras to β-actin and Rap to β-actin were calculated and expressed as the mean±SEM. Data were obtained from at least three independent experiments. * p<0.05 vs. no HB-EGF treatment.

FIG. 3 depicts HB-EGF-induced activation of ERK in PC12 cells exposed to oxygen glucose deprivation (OGD) injury. The intensity of immunoreactive bands on Western blots was quantified. The band intensity ratio of phosphorylated Erk1/2 to total Erk1/2 was calculated and expressed as the mean±SEM. Data were obtained from at least three independent experiments. * p<0.05 vs. normoxia.

FIG. 4 depicts the neuroprotective effect of HB-EGF on PC12 cells exposed to OGD. Panel (A) shows cell survival analyzed by MTT assay which quantifies surviving PC12 cells after 3 hours of OGD injury and 21 hours of return to normal glucose and oxygen levels. Panel (B) shows cell death measured by LDH (% of total) release into the medium after OGD injury. Cells treated with HB-EGF (20 ng/ml) had the growth factor added 16 hours prior to and during OGD injury. Cells that received AG1478 or PD98059 had the inhibitors added 30 minutes prior to HB-EGF addition. The experiment was repeated three times with similar results. *p<0.05.

FIG. 5 depicts the effect of HB-EGF on OGD-induced apoptosis in PC12 cells. In Panel (A), cells were grown under normal glucose/oxygen concentration (A), or were exposed to OGD (B-E). B) untreated cells; C) HB-EGF treated cells; D) cells that received AG1478 (EGFR inhibitor) 30 minutes prior to HB-EGF treatment; E) cells that received PD98059 (MAPK inhibitor) 30 minutes prior to HB-EGF treatment. Cells in the lower-left quadrant (LL), unstained for either Annexin V or PI, represent viable uninjured cells; cells in the lower right quadrant (LR), stained for Annexin V but not for PI, represent cells in the early or middle stages of apoptosis; cells in the upper-right quadrant (UR), positive for both Annexin and PI, represent later apoptotic or necrotic cells. The percentage of cells in each quadrant is shown at the bottom of each corresponding panel. F) quantification of apoptotic cells. *p<0.01 vs. untreated cells.

FIG. 6 depicts the effect of loss of HB-EGF on small bowel motility. Panel (A) is a photograph of the intestine of HB-EGF WT and KO mice 45 min after administration of methylene blue dye. The arrows show the most distal migration of the methylene blue dye. St, stomach; IC, ileocecal region. Panel (B) demonstrates that intestinal transit was assessed 45 min after administration of methylene blue and expressed as a percentage of total intestinal length. n=4 animals in each group. *p<0.01 compared to WT, Student's t test.

FIG. 7 depicts the effect of loss of HB-EGF gene expression on neurons in the myenteric plexus. Panel (A) provides representative photomicrographs of whole mount specimens from the ileal myenteric plexus of 4-week old WT and KO mice. Neurons are stained with the pan-neuronal marker PGP 9.5. Panel (B) provides quantification of the numbers of neuronal cells per ganglia in WT and KO mice. One hundred ganglia from HB-EGF KO or WT ileal segments were subjected to quantification, with counting of neuronal cells per ganglia. *p<0.01 compared to WT, Student's t test.

FIG. 8 depicts the effect of HB-EGF gene expression on neuronal nitric oxide synthase expression in neurons of the submucosal and myenteric plexuses. Panel (A) provides representative fluorescence photomicrographs of ileum from 4-week old WT and HB-EGF KO mice. Sections were stained with the neuronal cell marker HU and antibodies to nNOS. Panel (B) depicts a Western blot showing decreased nNOS expression in HB-EGF KO myenteric plexus and submucosal plexus.

### DETAILED DESCRIPTION

The following examples illustrate the invention wherein Example 1 describes a neonatal rat model of experimental NEC. Example 2 describes HB-EGF-induced neurite outgrowth. Example 3 demonstrates that HB-EGF increases MAP1B protein expression. Example 4 demonstrates that HB-EGF increases MAPK activation. Example 5 demonstrates that HB-EGF promotes cell survival after OGD injury. Example 6 demonstrates that HB-EGF knock out mice exhibit increased susceptibility to NEC. Example 7 demonstrates that HB-EGF is a chemoattractant for enteric crest cells. Example 8 demonstrates that gastric emptying and small bowel motility is impaired in HB-EGF knock out mice.

### EXAMPLES

### Example 1

### Neonatal Rat Model of Experimental Necrotizing Enterocolitis

The studies described herein utilize a neonatal rat model of experimental NEC. These experimental protocols were performed according to the guidelines for the ethical treatment of experimental animals and approved by the Institutional Animal Care and Use Committee of Nationwide Children's Hospital (#04203AR). Necrotizing enterocolitis was induced using a modification of the neonatal rat model of NEC initially described by Barlow et al. (J Pediatr Surg 9:587-95, 1974). Pregnant time-dated Sprague-Dawley rats (Harlan Sprague-Dawley, Indianapolis, Ind.) were delivered by C-section under CO₂ anesthesia on day 21.5 of gestation. Newborn rats were placed in a neonatal incubator for temperature control. Neonatal rats were fed via gavage with a formula containing 15 g Similac 60/40 (Ross Pediatrics, Columbus, Ohio) in 75 mL Esbilac (Pet-Ag, New Hampshire, Ill.), a diet that provided 836.8 kJ/kg per day. Feeds were started at 0.1 mL every 4 hours beginning 2 hours after birth and advanced as tolerated up to a maximum of 0.4 mL per feeding by the fourth day of life. Animals were also exposed to a single dose of intragastric lipopolysaccharide (LPS; 2 mg/kg) 8 hours after birth, and were stressed by exposure to hypoxia (100% nitrogen for 1 minute) followed by hypothermia (4° C. for 10 minutes) twice a day beginning immediately after birth and continuing until the end of the experiment. In all experiments, pups were euthanized by cervical dislocation upon the development of any clinical signs of NEC. All remaining animals were sacrificed at the end of experiment at 96 hours after birth.

The HB-EGF used in all experiments was GMP-grade human mature HB-EGF produced in P. pastoris yeast (KBI BioPharma, Inc., Durham, N.C.). EGF was produced in E. coli and purchased from Vybion, Inc. (Ithaca, N.Y.).

To assess the histologic injury score, immediately upon sacrifice, the gastrointestinal tract was carefully removed and visually evaluated for typical signs of NEC including areas of bowel necrosis, intestinal hemorrhage and perforation. Three pieces each of duodenum, jejunum, ileum, and colon from every animal were fixed in 10% formalin for 24 hours, paraffin-embedded, sectioned at 5 μm thickness, and stained with hematoxylin and eosin for histological evaluation of the presence and/or degree of NEC using the NEC histologic injury scoring system described by Caplan et al. (Pediatr. Pathol. 14:1017-28, 1994). Histological changes in the intestines were graded as follows: grade 0, no damage; grade 1, epithelial cell lifting or separation; grade 2, necrosis or sloughing of epithelial cells to the mid villus level; grade 3, necrosis of the entire villus; and grade 4, transmural necrosis. All tissues were graded blindly by two independent observers. Tissues with histological scores of 2 or higher were designated as positive for NEC.

Fisher's exact test was used for comparing the incidence of NEC between groups with no adjustments made for multiple comparisons. P-values less than 0.05 were considered statistically significant. All statistical analyses were performed using SAS, (version 9.1, SAS Institute, Cary, N.C.).

When the pups are exposed to stress in the absence of HB-EGF, about 65% of the pups suffered from NEC at grades 2-4. However, only about 23.8% of the pups exposed to stress in combination with administration of HB-EGF suffered from NEC at grade 2-4.

### Example 2

### HB-EGF Induces Neurite Outgrowth

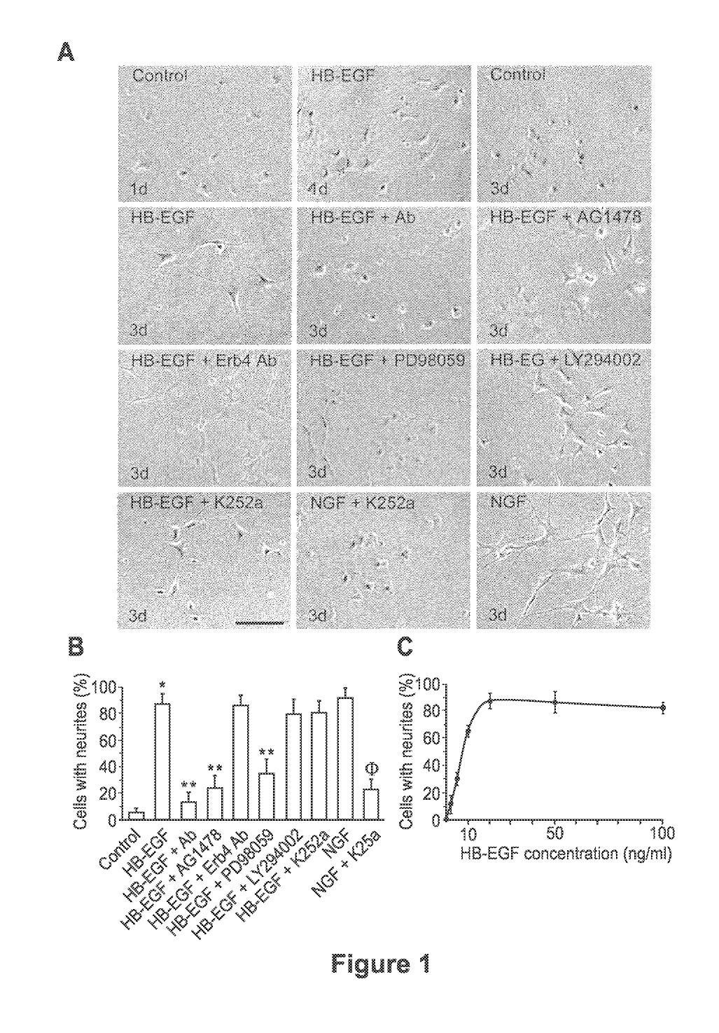

Neurite outgrowth represents a morphological change in neuronal tissue that results in synaptic formation both during development and during the axon pathfinding that occurs after nerve injury (Kyoto et al. Brain Res; 1186:74-86., 2007). The ability of HB-EGF to affect neurite outgrowth in PC12 cells was investigated. HB-EGF-induced PC12 cell differentiation, as demonstrated by significant neurite outgrowth extension as early as 1 day after HB-EGF addition (FIG. 1A).

To measure neurite outgrowth, 4×10³ PC12 cells were seeded in each well of an 8-well culture slide chamber coated with poly-D-lysine and lamine (BD Biosciences, Bedford, Mass., USA) and starved with serum free DMEM for 16 hours. After addition of HB-EGF (20 ng/ml) or NGF (50 ng/ml positive control), cells were incubated for an additional 24 hours and 72 hours, and random photographs were taken for quantification of neurite outgrowth. Other agents such as AG1478 (1 mmol; selective EGF receptor kinase inhibitor) (Cayman Chemical, Ann Arbor, Mich., USA), monoclonal antibody against the ErbB-4 extracellular domain (MAb-3, colone H72.8, 30 μg/ml, NeoMarker, Fremont, Calif., USA), PD98059 (20 μmol; selective inhibitor of MAP kinase kinase) (Calbiochem, Gibbstown, N.J., USA), U0126 (10 μmol; selective inhibitor of Erk1/2, Calbiochem), LY294002 (50 μmol; inhibitor of phosphoinositide 3-kinase (PI3K) pathway, Calbiochem) or K252a (1 μmol; Trk tyrosine kinase receptor inhibitor) (Sigma-Aldrich, Saint Louis, Mich., USA) were added 30 minutes prior to HB-EGF treatment. The proportion of neurite-bearing cells was counted using an inverted microscope and phase contrast microscopy. Cell processes longer than the cell body diameter were counted as neurites, with neurites identified and counted in 100 cells per photograph. Three independent experiments were performed. To investigate whether neurite outgrowth was specifically induced by HB-EGF, HB-EGF (20 ng/μl) was pre-incubated with neutralizing HB-EGF antibodies (1 μg/μl; R&D Systems Inc., Minneapolis, Minn., USA) for 60 minutes at 37° C., and then the neutralized HB-EGF was added to the medium in the PC12 neurite outgrowth assay. Addition of the neutralizing HB-EGF antibody significantly decreased neurite outgrowth (FIG. 1A). HB-EGF-induced neurite outgrowth in PC12 cells was determined to be dependent upon activation of the EGF receptor (EGFR). PC12 cell were pretreated with the EGF receptor kinase inhibitor AG1478 for 30 minutes prior to HB-EGF stimulation. HB-EGF-induced neurite outgrowth was significantly inhibited by the addition of AG1478 (FIG. 1A). On the other hand, blockage of the ErbB-4 receptor subtype using a neutralizing monoclonal antibody did not alter HB-EGF-induced neurite outgrowth. In addition, blockage of Trk tyrosine kinase receptor activation with K252a did not reduce the effect of HB-EGF on neurite outgrowth (FIG. 1A).

Since activation of the MAPK pathway has been reported to play a critical role in neuronal cell differentiation after growth factor stimulation, whether HB-EGF-induced neurite outgrowth was dependent on the MAPK pathway was investigated. The Erk kinase inhibitor PD98059 markedly reduced HB-EGF-induced neurite outgrowth (FIG. 1A). Similar to the effects of PD98059, MAPK inhibition with U0126 (selective inhibitor of Erk1/2) significantly blocked HB-EGF-induced neurite outgrowth as well. These observations suggest that activation of MAPK is crucial for HB-EGF-induced neurite outgrowth. However, the PI3K inhibitor LY2942002 did not compromise the effect of HB-EGF on PC12 neurite outgrowth. (FIG. 1A).

To quantify neurite extension, differentiated PC12 cells containing at least one dendrite longer than the cell body after a 3 day incubation in the presence or absence of HB-EGF were counted. Compared to non-HB-EGF-treated control cells, substantial neurite outgrowth was observed in HB-EGF treated PC12 cells (87.8±7.9% vs. 5.8±3.6%; p<0.01) (FIG. 1B). AG1478 and PD98059 significantly reduced the rate of neurite extension induced by HB-EGF to 24.3±9.6 and 35.8±9.55% receptively, while K252a or LY2942002 had no effect, suggesting that HB-EGF-induced neurite outgrowth was dependent upon EGFR activation and the MAPK pathway rather than the Trk tyrosine kinase or PI3K pathways. Of particular note is that the Trk tyrosine kinase pathway has been reported to be activated in differentiated PC12 cells stimulated by NGF.

The effect of HB-EGF on neurite outgrowth in PC12 cell was found to be dose dependent (FIG. 1C). After 3 day incubation with HB-EGF, maximal neurite extension was observed with addition of 20 ng/ml HB-EGF.

### Example 3

### HB-EGF Increases MAP1B Protein Expression

Microtubule associated protein 1b (MAP1b) is a neuronal cytoskeletal marker with predominant expression in the developing nervous system, which is frequently used as a marker for neuronal cell sprouting (Keating et al., Dev Biol; 162:143-531994; Goold et al., J Cell Sci; 114:4273-84, 2001; Fischer et al., Mol Cell Neurosci; 2:39-51, 1991; Mansfield et al., J Neurocytol; 20:1007-22, 1991). PC12 cells were treated with HB-EGF (20 ng/ml) for 24 hours, followed by immunocytochemical detection of MAP1b using anti-MAP1b monoclonal antibodies.

PC12 cells were seeded in 8-well culture slides coated with poly-D-lysine/laminin and were incubated with or without HB-EGF (20 ng/ml). After 24 hours, cells were fixed with 4% paraformaldehyde in 0.1M PBS for 30 minutes, and blocked with 10% goat serum, 0.1% Triton X100/PBS for 30 min. After incubation with primary antibody (anti-MAP1b mAb) (Sigma, Saint Louis, Mich., USA) for 2 hours, cells were rinsed with PBS and incubated with Cy2-labeled secondary antibody (Molecular Probes, Billerica, Mass., USA) for 1 hour. Propidium iodide (Invitrogen, Carlsbad, Calif., USA) was used to visualize nuclei. Fluorescent staining was examined using a Zeiss AxioSkop 2 Plus microscope (Carl Zeiss Inc., Thornwood, N.Y., USA).

This study demonstrated that HB-EGF significantly increased MAP1b immunostaining in the cytoplasm and dendrites of PC12 cells. The elevated protein expression of MAP1b confirms that HB-EGF promotes neuronal differentiation of PC12 cells.

### Example 4

### HB-EGF Increases MAPK Activation

Mitogen activated protein kinase (MAPK) activation is necessary for growth factor-induced neurite outgrowth in PC12 cells (Patapoutian et al., Curr Opin Neurobiol 11:272-80 2001). Activation of the MAPK pathway is involved in the reorganization of microtubules towards the future direction of neurite outgrowth under normal conditions and after cellular injury (Morishima-Kawashima et al., Mol Biol Cell 1996; 7:893-905, 1996; Goold et al., Mol Cell Neurosci; 28:524-34, 2005).

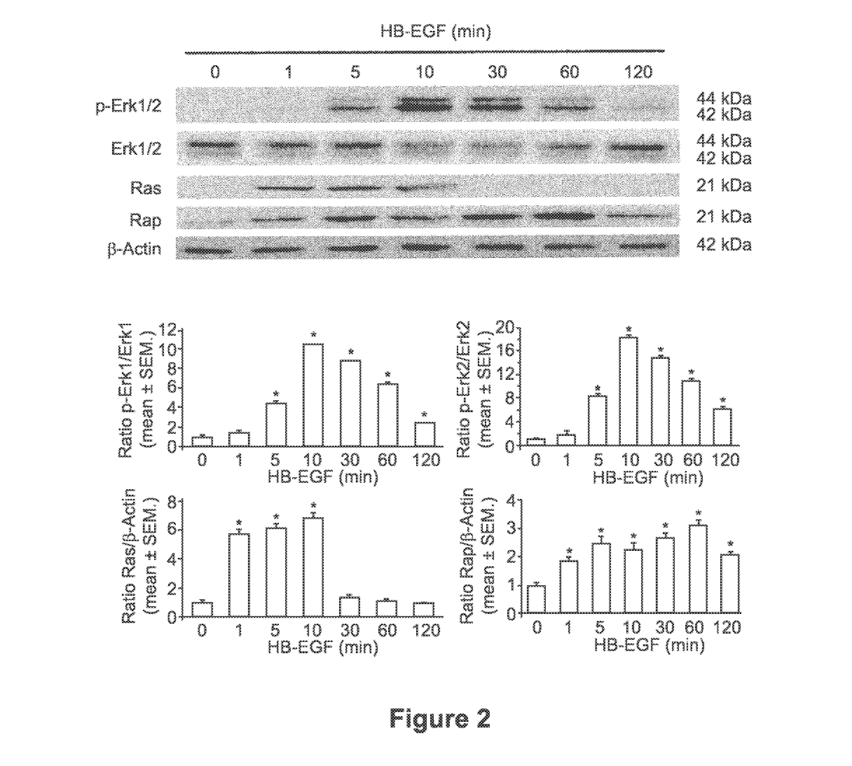

Since HB-EGF-induced neurite outgrowth was inhibited by PD98059 (FIG. 1B), the role of MAPK activation in this pathway was confirmed using immunoblot analysis to examine the ability of HB-EGF to affect phosphorylation of Erk1/2. Cells were stimulated with HB-EGF (20 ng/ml) for various times. Cell lysates were separated by SDS-PAGE and analyzed by immunoblotting. Activated MAPK was specifically recognized by a rabbit anti-phosphorylated Erk1/2 antibody. The blot was then reprobed with a rabbit antibody to total Erk1/2. To detect activated Ras and Rap, lysates were clarified by centrifugation, and supernatants were collected and incubated with glutathione-Sepharose beads coupled to C-RafRBD/GST or RalGDSRBD/GST. After incubation, the samples were separated by SDS-PAGE and analyzed by Western blotting with mouse anti-Ras or anti-Rap1 antibodies. The induction of phosphorylation of Erk 1/2 by HB-EGF appeared at 1 minute, peaked at 10 to 30 minutes, and lasted for at least for 2 hours (FIG. 2). This pattern of Erk activation is similar to NGF-induced Erk signaling in PC12 cells (Peraldi et al., Endocrinology, 132:2578-85, 1993).

Two distinct pathways are involved in the activation of Erk: the small G protein Ras is required for the initial activation of Erk and the small G protein Rap1 is required for the sustained activation of Erk (Powers et al., Cell Tissue Res; 295:21-32 1999; York et al. Nature 92:622-6, 1998). Therefore, studies were carried out to investigate whether HB-EGF activates Ras and Rap1 in PC12 cells. Ras activation was detected within 1 minute after HB-EGF stimulation, lasted for 10 minutes, and then dramatically decreased thereafter. Rap1 activation was induced within 1 minute and lasted for at least 2 hours, a pattern that matches the sustained activation of Erk1/2. These results suggest that HB-EGF activates Ras and Rap1, leading to the activation of Erk1/2 in PC12 cells.

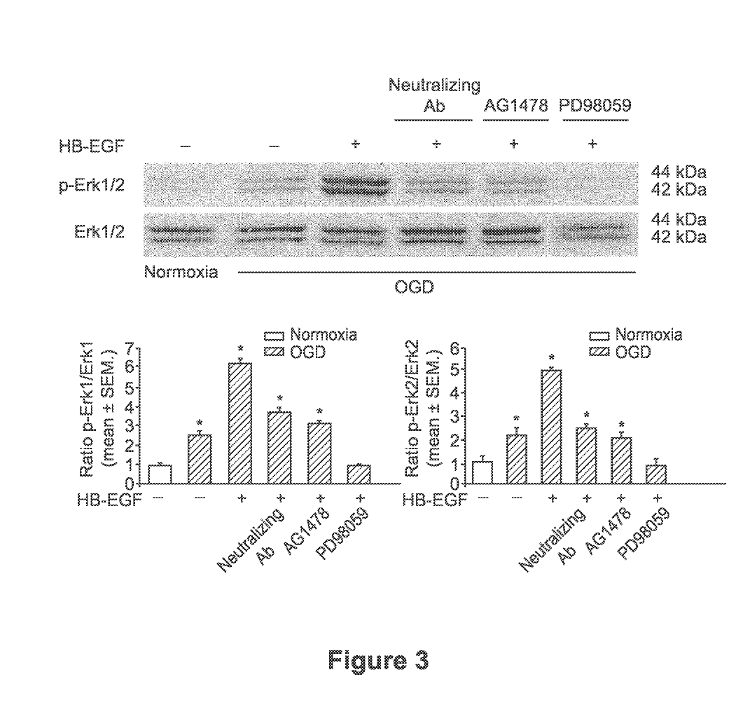

MAPK activation promotes neuronal cell survival and inhibits apoptosis after ischemic injury (Bonni et al. Science 286:1358-62, 1999b; Zhou et al., Mol Ther; 12:402-12, 2005). Therefore, the ability of HB-EGF to activate the MAPK pathway by detecting phosphorylation of Erk1/2 in PC12 cells exposed to OGD injury was investigated. PC12 cells were exposed to oxygen glucose deprivation (OGD) for 3 hours, followed by addition of glucose and renewal of normoxia for an additional 21 hours. Some cells received neutralized HB-EGF by preincubating HB-EGF (20 ng/μl) with neutralizing HB-EGF antibodies (1 μg/μl) for 60 minutes at 37° C. Cells were then exposed to OGD for 3 hours followed by return to normoxia and normal glucose levels for 21 hours. Cell lysates were then collected for evaluation of Erk activation by immunoblotting using anti-phospho Erk1/2. Pan-Erk1/2 was used to verify equal protein loading in all lanes. The intensity of immunoreactive bands on Western blots was quantified Immunoblot analysis of protein extracts form PC12 cells 24 hours after oxygen glucose deprivation (OGD) (Tabakman et al., Ann N.Y. Acad. Sci. 1053:84-9 (2005), Hu et al., Neurosci. Lett. 423:35-40 (2007)) injury revealed enhanced Erk1/2 phosphorylation following addition of HB-EGF (FIG. 3). The increase in Erk1/2 phosphorylation induced by HB-EGF was suppressed by preincubation of HB-EGF with neutralizing anti-HB-EGF antibodies or by addition of AG1478 (EGFR inhibitor) or PD98059 (MAPK inhibitor). These results show that HB-EGF is able to activate the MAPK pathway in PC12 cells even in an environment of neuronal cell injury. Notably, the protective effect of HB-EGF on injured PC12 cells is specific and EGFR dependent.

### Example 5

### HB-EGF Promotes Cell Survival after OGD Injury

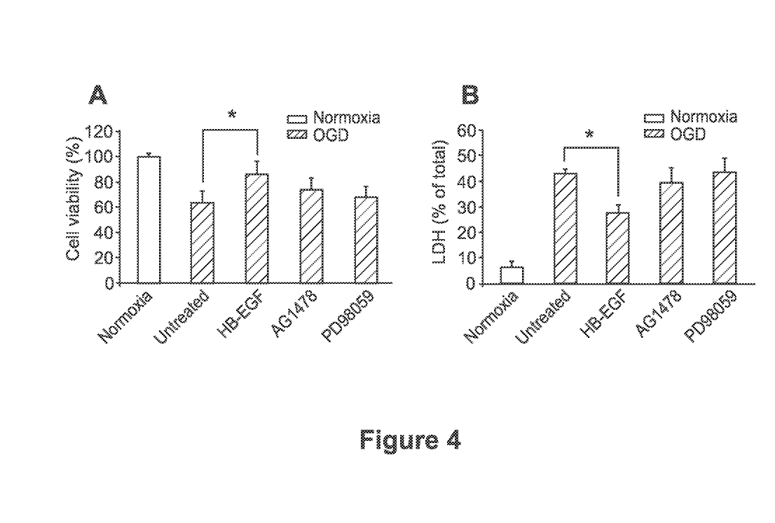

The neuroprotective effect of HB-EGF was investigated on pheochromocytoma neuronal cells (PC 12) exposed to injury using a model of oxygen glucose deprivation (OGD) (Tabakman et al., Ann N.Y. Acad. Sci. 1053:84-9 (2005), Hu et al., Neurosci. Lett. 423:35-40 (2007)). PC 12 cells were exposed to OGD for 3 hours, followed by addition of glucose and renewal of oxygen for an additional 24 hours, which caused further reoxygenation injury. This system provided an in vitro model that mimics ischemia reperfusion injury. Some cells received HB-EGF or EGF or NGF (a well recognized neuroprotective growth factor) before and during the OGD insult. The MTT assay was used to detect viable PC 12 cells 24 hours after OGD insult (Mosmann, Journal of immunological methods 65 (1-2): 55-63, 1983). Treatment of PC 12 cells with HB-EGF led to significantly increased cell viability (FIG. 4A). This observation suggests that HB-EGF has a neuroprotective effect. In addition, HB-EGF treatment of PC 12 cells resulted in increased phosphorylation of Erk 1/2 under basal conditions and after OGD injury to the cells. This suggests that HB-EGF-induced neuronal cell protection is related, at least in part, to MAPK activation.

Addition of EGF receptor kinase-inhibitor AG1478 or Map kinase kinase inhibitor PD98059 suppressed the HB-EGF-mediated neuroprotective effects. Under conditions of cellular necrosis or apoptosis, cells lose cell membrane stabilization and thereafter release LDH. In addition, LDH leakage was quantified as a parameter of cell membrane integrity. LDH release was increased in PC12 cells subjected to OGD injury, whereas addition of HB-EGF to PC12 cells exposed to OGD injury led to decreased LDH release (FIG. 4B). Again, AG1478 or PD98059 decreased the neuroprotective effects of HB-EGF.

Apoptosis is the main process involved in OGD-induced cell death in PC12 cells. PC12 cell apoptosis was assessed using the Vybrant Apoptosis Assay (Invitrogen, Carlsbad, Calif., USA). Cells were seeded in 100 mm culture dishes coated with poly-D-lysine/laminin at a density of 1×10⁶ cells/well. After 12 hours of low serum (1% FBS) starvation, some cells were pretreated with HB-EGF (20 ng/ml) for 16 hours prior to OGD injury. Twenty-four hours after OGD injury, cells attached to the plates and floating dead cells were harvested and resuspended in binding buffer. FITC-Annexin V (1 mg/ml) was then added to the resuspended cells with incubation for 10 minutes at 37° C. Cells were resuspended in propidium iodide (PI) solution and incubated in the dark for 30 minutes at room temperature. Stained cells were analyzed using a BD LSR II flow cytometer (BD Biosciences, San Jose, Calif., USA). Chemical inhibitors (AG1478, PD98059) were added to the culture medium 30 min prior to HB-EGF treatment.

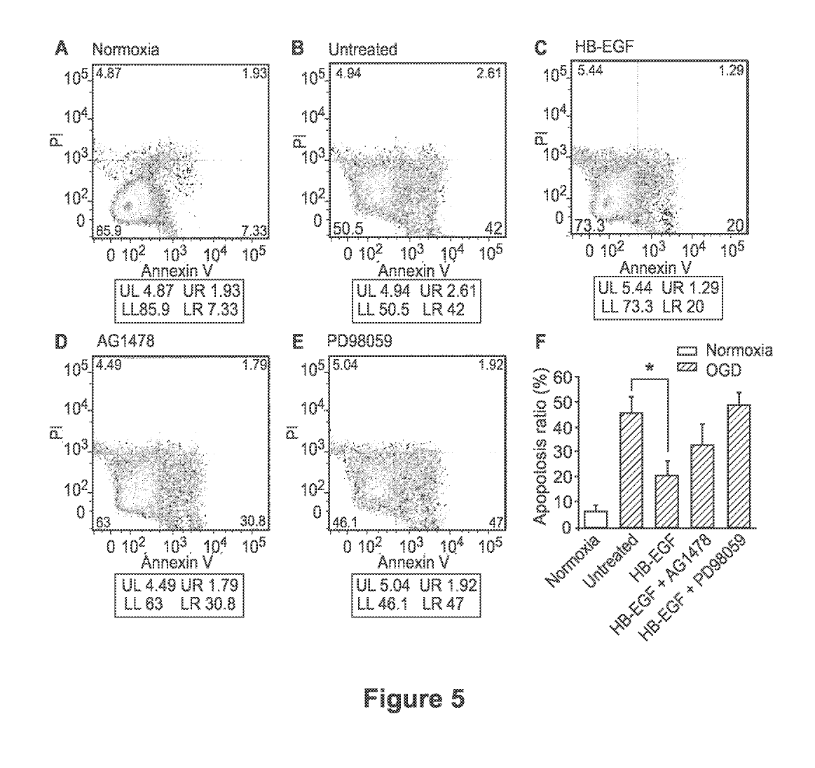

The effect of HB-EGF on PC12 cell apoptosis upon exposure of the cells to OGD injury was also examined. HB-EGF was added to cultured PC12 cells 16 hours prior to OGD injury. After 3 hours of OGD and 21 hours of return to normal glucose and oxygen levels, HB-EGF significantly decreased the percentage of apoptotic cells compared with untreated control cells (20.9±5.9 vs. 45.4±4.67; p<0.01) (FIG. 5). Addition of PD98059 completely abolished the neuroprotective effects of HB-EGF while AG1478 partially blocked HB-EGF-mediated neuroprotection.

The neuroprotective effect of HB-EGF was also investigated on PC 12 cell neurite outgrowth. Neurite outgrowth represents a morphological change in neuronal tissue that results in synaptic formation both during development and during the axon pathfinding that occurs after nerve injury (Tom et al., J. Neurosci. 24:6531-9 (2004); Kyoto et al., Brain Res. 1186:74-86 (2007)) HB-EGF treated PC 12 cells had significant induction of neurite outgrowth, aggregation of the cells, and formation of nerve fiber networks compared to non-treated cells (FIG. 2). HB-EGF treated PC 12 cells had significantly increased neurite length. In addition, PC 12 cells aggregated and formed a fiber network in the presence of HB-EGF. Furthermore, HB-EGF significantly induced down-regulated NRP-1 expression—an inhibitor of neurite outgrowth. This suggests that HB-EGF promotes neurite outgrowth, at least in part, by down regulating NRP-1 expression.

### Example 6

### HB-EGF Knock Out Mice Exhibit Increased Susceptibility to NEC

The role of endogenous HB-EGF gene expression in susceptibility to intestinal injury and the preservation of gut barrier function in a newborn mouse model of experimental NEC using HB-EGF Knock Out (KO) mice was investigated. HB-EGF knock out (KO) mice on a C57BLI6J×129 background and HB-EGF WT C57BL/6J×129 mice as described by Jackson et al. (EMBO J. 22: 2704-2716, 2003) were used. In the HB-EGF KO mice, HB-EGF exons 1 and 2 were replaced with PCK-Neo, thus deleting the signal peptide and propeptide domains. The desired targeting events were verified by Southern blots of genomic DNA and exon-specific polymerase chain reaction, with Northern blots confirming the absence of the respective transcripts.

NEC was induced using the experimental model described in Example 1 as modified for mice as described by Jilling et al. (J. Immunol. 177: 3273-3282, 2006). Pregnant time-dated mice were delivered by C section under inhaled 2% Isofturane (Butler Animal Health, Dublin, Ohio) anesthesia on day 18.5 of gestation. Newborn mouse pups were placed in an incubator (37° C.) and fed via gastric gavage with formula containing 15 g Similac 60/40 (Ross Pediatrics, Columbus, Ohio) in 75 mL Esbilac (Pet-Ag, New Hampshire, Ill.), providing 836.8 kJ/kg per day. Feeds were started at 0.03 mL every 3 hours beginning 2 hours after birth and advanced as tolerated up to a maximum of 0.05 mL per feeding by the fourth day of life. Animals were stressed by exposure to hypoxia (100% nitrogen for 1 minute) followed by hypothermia (4° C. for 10 minutes) once a day beginning immediately after birth until the end of the experiment. Exposure of pups to hypoxia, hypothermia and hypertonic feeds will subsequently be referred to herein as exposure to “stress”.

To investigate the effects of HB-EGF loss-of-function on susceptibility to NEC, HB-EGF WT pups (n=19) and HB-EGF KO pups (n=31) were exposed to experimental NEC. An additional group of HB-EGF KO pups (n=33) were exposed to experimental NEC as described, but received HB-EGF (800 pg/kg/dose) added to each feed (starting 2 hours after birth). The HB-EGF used was Good Manufacturing Practice (GMP) grade human mature HB-EGF produced in Pichia pastoris yeast (Trillium Therapeutics, Inc., Toronto, Canada). In all experiments, pups were euthanized upon development of clinical signs of NEC (abdominal distention, bloody bowel movements, respiratory distress, and lethargy). Remaining animals were sacrificed 96 hours after birth.

### Histologic Injury

Upon sacrifice, the gastrointestinal tract was carefully removed and visually evaluated for signs of NEC (areas of bowel necrosis, intestinal hemorrhage, perforation). Three pieces of duodenum, jejunum, ileum, and colon from every animal were fixed in 10% formalin for 24 hours, paraffin-embedded, sectioned at 5 μm thickness, and stained with hematoxylin and eosin for histological evaluation of the presence and/or degree of NEC using the NEC histologic injury scoring system described by Caplan et al. (Pediatric Pathol. 14: 1017-1028, 2007) Histological changes were graded as follows: grade 0: no damage; grade 1: epithelial cell lifting or separation; grade 2: sloughing of epithelial cells to the mid villus level; grade 3: necrosis of the entire villus; and grade 4: transmural necrosis. Tissues were graded blindly by two independent observers. Tissues with histological scores of 2 or higher were considered positive for NEC.

Histologic analyses revealed that HB-EGF WT mouse pups had an incidence of NEC of 53%, with grade 2 injury seen in 100% of the animals that developed NEC. HB-EGF KO mice had a significantly increased incidence of NEC of 80% (p=0.04), with histopathologic changes ranging from moderate, mid-level villous necrosis (grade 2) to severe necrosis of the entire villous (grade 3). Of the 80% of pups that developed NEC, 48% had grade 2 injury and 32% had grade 3 injury. HB-EGF KO pups exposed to stress but with HB-EGF (800 μg/kg/dose) added to the feeds showed a significant decrease in the incidence of NEC to 45% compared to stressed pups that were not treated with HB-EGF (p=0.004). In addition to a decreased incidence of NEC, supplementation of HB-EGF to the formula of HB-EGF KO pups resulted in decreased severity of NEC. Of the 45% of HB-EGF-treated pups that developed NEC, 44% had grade 2 injury and only 3% had grade 3 injury.

### Gut Barrier Function

Intestinal permeability was also examined to determine gut barrier function in HB-EGF WT and HB-EGF KO mice exposed to experimental NEC. Fluorescein isothiocyanate (FITC)-labeled dextran molecules (molecular weight, 73 kDa) (Sigma-Aldrich Inc, St Louis, Mo.) was used as a probe to examine gut barrier function. Previous studies by others have shown that use of 73-kDa dextran molecules results in a reliable assessment of mucosal perturbations 4 hours after enteral administration (Caplan et al. Gastroenterology 117:577-583, 1999). In this experiment, FITC-labeled dextran molecules (750 mg/kg) were administered via orogastric tube to mouse pups. After 4 hours, blood was collected and plasma FITC-dextran levels were measured using spectrophotofluorometry (Molecular Devices, SpectraMax M2, Sunnyvale, Calif.). The amount of dextran in the plasma was calculated based on standard dilution curves of known dextran concentrations. The mouse pups were divided into 4 groups as follows: 1) WT mice that received intragastric FITC-dextran immediately after birth with no exposure to stress (n=15); 2) HB-EGF KO mice that received intragastric FITC-dextran immediately after birth with no exposure to stress (n=17); 3) HB-EGF WT mice that received intragastric FITC dextran after 24 hours of stress (n=13); and 4) HB-EGF KO mice that received intragastric FITC dextran after 24 hours of stress (n=10).

The Chi-square test was used for comparing the incidence of NEC between groups. Serum concentrations of FITC-dextran were compared using the Student's t test. p-values less than 0.05 were considered statistically significant. All statistical analyses were performed using SAS software (Version 9.1, SAS Institute, Cary, N.C.).

Under basal, non-stressed conditions immediately after birth, HB-EGF KO pups had significantly increased serum FITC-dextran levels compared to HB-EGF WT pups (179.73±58.43 μg/ml vs. 47.79±14.39 μg/ml; p=0.04). After 24 hours of exposure to stress, HB-EGF WT mice had increased serum FITC-dextran levels compared to HB-EGF WT mice under basal conditions (119.86±36.39 μg/ml vs. 47.79±14.39μ/ml; p=0.00003). On the other hand, HB-EGF KO pups exposed to stress for 24 hours had a much smaller increase in serum FITC-dextran levels compared to KO mice under basal conditions (190.70±61.54 μg/ml vs. 179.73±58.43 μg/ml), but still had much higher serum FITC-dextran levels compared to WT mice exposed to stress for 24 hours (190.70±61.54 μg/ml vs. 119.86±36.39 μg/ml; p=0.3). The FITC-dextran serum levels in WT animals after birth are low, indicating intact intestinal bather function, but as the animals are exposed to stress for 24 hours there is an increase in serum FITC-dextran levels indicating damage to the mucosal barrier. HB-EGF KO mice have increased FITC-dextran serum levels immediately after birth and maintain high serum levels at the 24 hour time point as well, suggesting a baseline deficit in gut barrier function that may explain, in part, their increased susceptibility to NEC.

These experiments demonstrate that newborn HB-EGF KO mice have increased susceptibility to experimental NEC, and show that they have increased intestinal permeability under both basal and stressed conditions. The effects of lack of endogenous HB-EGF on the intestine can be compensated for by administration of exogenous enteral HB-EGF. These findings support the concept of administration of HB-EGF to patients with or at risk of developing NEC in order to prevent the progression of or development of the disease.

Studies in critically ill adults have shown that impairment of mucosal barrier function with overgrowth of pathogenic bacteria in the gastrointestinal tract enhances translocation of bacteria and endotoxin, resulting in a septic inflammatory response and multiorgan failure (Deitch, Arch Surg 125:403-404, 1990; Hadfield et al. Am. J. Respir. Crit. Care Med. 152:1545-1548, 1995). Plena-Spoel et al. (J. Pediat. Surg. 36: 587-592, 2001) evaluated changes in intestinal permeability in 13 children with NEC compared to 10 control patients undergoing surgery by measuring lactulose to rhamnose ratios in urine samples. They found that lactulose to rhamnose ratios in NEC patients were increased for prolonged periods of time, with high peaks seen in patients with sepsis, indicative of gut barrier failure. Control patients had increased intestinal permeability only in the first days after surgery, which normalized rapidly afterwards. Beach et al. (Arch. Dis. Childhood, 57: 141-145, 1982) observed increased intestinal permeability during the first week of life in neonates of gestational age 31-36 weeks, while Weaver (Arch. Dis. Childhood, 59: 236-241, 1984) showed that premature newborns born prior to 34 weeks gestation exhibited higher intestinal permeability than more mature newborns. The impaired gut barrier function of premature babies under basal conditions may be similar to the impaired intestinal permeability reported here in newborn HB-EGF KO mice under basal conditions. When HB-EGF expression is decreased or absent, as in the intestine of neonates afflicted with NEC or in HB-EGF KO mice, gut barrier function is impaired, which may contribute to bacterial translocation leading to a systemic inflammatory response.

The results of the current study, demonstrating increased intestinal injury and increased intestinal permeability in HB-EGF KO mice exposed to experimental NEC, support the contention that HB-EGF expression is important in protection of the intestines from NEC. The fact that administration of exogenous HB-EGF to HB-EGF KO mice protects the intestines from experimental NEC supports the clinical administration of HB-EGF to patients with or at risk of developing NEC in an effort to treat or prevent the disease.

### Example 7

### HB-EGF is a Chemoattractant for Enteric Neural Crest Cells

Whole-mount immunohistochemistry of the hindgut, before and after birth, using a marker for nerve cells was carried out in wild-type and HB-EGF KO mice. This demonstrated that during development, HB-EGF KO mice have significantly delayed migration of neural crest cells compared to wild type mice, with significantly fewer ganglia in the KO mice compared to wild type mice. One month after birth, HB-EGF KO mice had significantly reduced neuronal cells in the myenteric plexuses where the ganglia appeared empty when compared to the neurons of wild-type mice.

### Example 8

### Gastric Emptying and Small Bowl Motility Impaired in HB-EGF Knock Out Mice

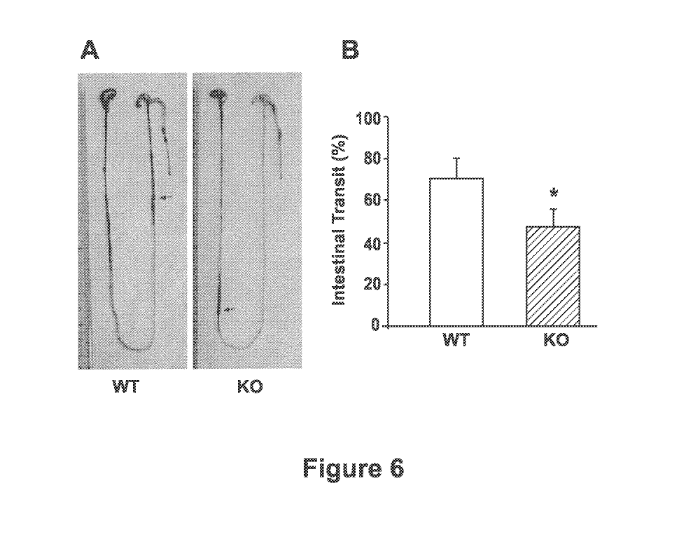

Evidence suggests that NEC is due to an inappropriate inflammatory response of the immature gut to an undefined insult (Henry & Moss, Annu Rev Med 2008). The underdeveloped enteric nervous system of the premature infant may predispose prematures to NEC (Berseth et al., J Pediatr. 115:646-51 (1989); Bernat et al., J. Lipid Medial. 5:41-48 (1992)). In addition, most NEC patients develop long-term gastrointestinal dysfunction with decreased intestinal motility upon recovery from NEC (Neu, Pediatr. Clin. North Am. 43:409-32 (1996); Dudgeon et al., J Pediatr. Surg. 8:607-14 (1973)). It is hypothesized that lack of HB-EGF leads to abnormal development of the enteric nervous system and impaired gastrointestinal motility. The use of oral gavage of methylene blue, a dye that is not absorbed in the GI system, demonstrated that HB-EGF KO mice have significantly delayed gastric emptying (FIG. 6A) and small bowel transit time (FIG. 6B) compared to WT mice. This suggests that HB-EGF plays an important role in promoting GI motility.

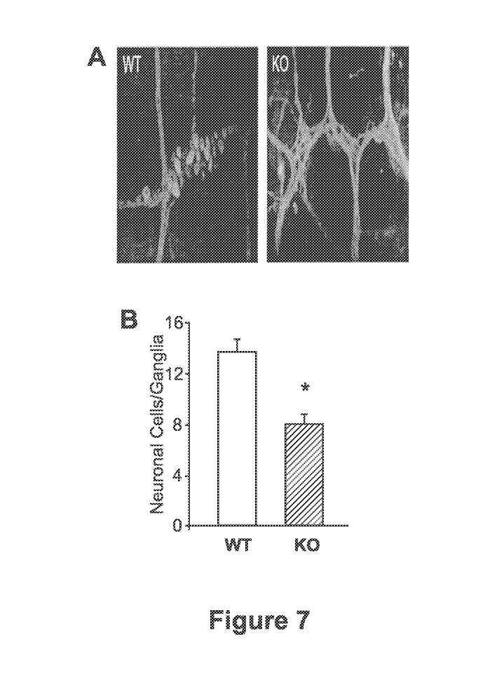

The morphologic features of enteric neurons isolated from the myenteric plexuses of HB-EGF KO and WT mice were investigated. This study demonstrated that the intestinal myenteric plexus of HB-EGF KO mice had a decreased number of neuronal cells. Using whole mount specimens of mouse ileal myenteric plexuses, the number of neurons contained in the myenteric plexus as identified using PGP 9.5 immunostaining were quantified. The average number of neurons was significantly decreased in HB-EGF KO mice compared to WT mice (FIG. 7 A, B). In addition, hypertrophied nerve fibers were noted in HB-EGF KO mice (FIG. 7A). These results suggest that absence of HB-EGF is associated with myenteric neuronal degeneration.

Deletion of the HB-EGF gene also decreases neuronal nitric oxide synthase (nNOS) production in myenteric plexus ganglia. Nitric oxide (NO) is a diffusible unstable gas that plays a role in neuronal development, plasticity, and neurite remodeling (Reyes-Harde et al, J Neurophysiol; 82:1569-76 (1999); Gally et al., Proc Natl. Acad. Sci. U.S.A. 87:3547-51 (1990)). NO is also a major neurotransmitter in the gastrointestinal tract that regulates the muscular tone of the intestine and modulates peristalsis (Takahashi, J. Gastroenterol; 38:421-30 (1990), Spencer et al., J Physiol. 530:295-306 92001), Ciccocioppo et al., J Pharmacol. Exp Ther.; 270:929-37 (1994)).

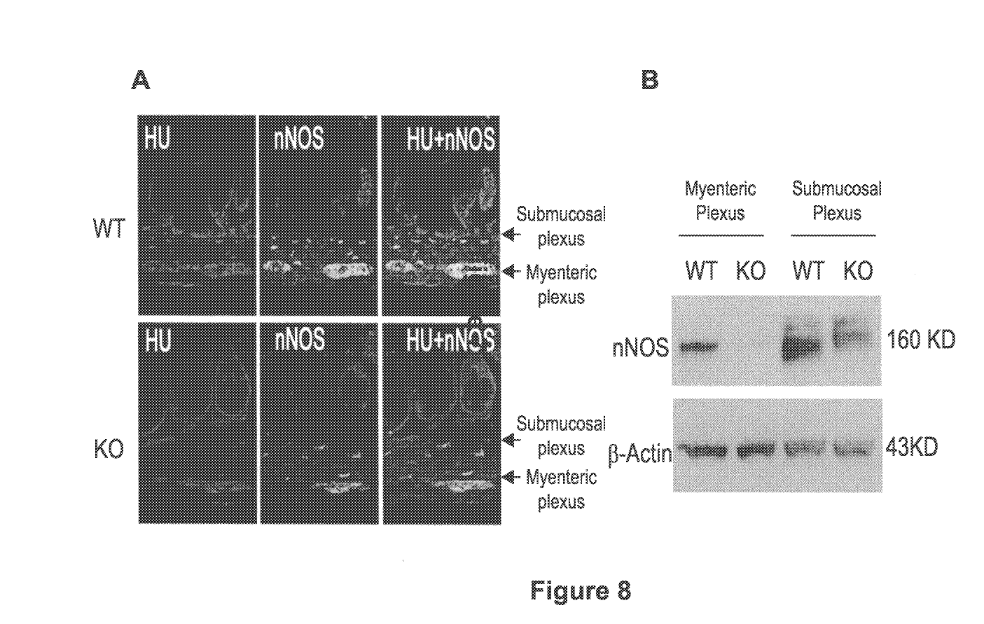

NO synthesis in the ENS is mediated by neuronal nitric oxide synthase (nNOS). Compromised nNOS function is associated with diminished local production of NO, which may lead to degenerative ENS neuropathy and disordered gastrointestinal motility. In addition, normal expression of nNOS suppresses inducible NOS (iNOS), (Qu et al., B. Faseb J; 15:439-46 (2001)) an enzyme involved in the inflammatory response. nNOS expression in HB-EGF WT and KO mice was examined by immunohistochemistry and Western Blotting. nNOS expression was significantly decreased in HB-EGF KO myenteric plexus and submuosal plexus ganglia (FIG. 8). This finding suggests that decreased nNOS expression in HB-EGF KO mice impairs the normal development of the ENS, and may make the intestine more vulnerable to inflammatory processes such as NEC.

1. A method of increasing intestinal motility in a patient suffering from intestinal injury comprising administering an EGF receptor agonist in an amount effective to increase intestinal motility.

2. (canceled)

3. A method of protecting neurons within the enteric nervous system (ENS) in a patient suffering from intestinal injury comprising administering an EGF receptor agonist in an amount effective protect neurons within the ENS.

4. A method of inducing neurite growth within the enteric nervous system (ENS) in a patient suffering from intestinal injury comprising administering an EGF receptor agonist in an amount effective to induce neurite growth.

5. The method of claim 4, wherein the EGF receptor agonist is a HB-EGF product.

6. The method of claim 5, wherein the HB-EGF product comprises amino acids of 74-148 of SEQ ID NO: 2.

7. The method of claim 4, wherein the EGF receptor agonist is an EGF product.

8. The method of claim 7, wherein the EGF product comprises amino acids 1-53 of SEQ ID NO: 4.

9. The method of claim 4, wherein the intestinal injury is necrotizing enterocolitis, hemorrhagic shock and resuscitation, ischemia/reperfusion injury, intestinal inflammatory conditions or an intestinal infections.

10. The method of claim 4, wherein the patient is suffering from Hirschprung's Disease, intestinal dysmotility disorders, intestinal pseudo-obstruction (Ogilvie's Syndrome), irritable bowel syndrome or chronic constipation.

11. The method of claim 9, wherein the intestinal injury is caused by necrotizing enterocolitis (NEC).

12. The method of claim 4, wherein the patient is an infant.
